SEQUENCE LISTING (6 sequences)

SEQ ID NO: 1 - DNA, 2766 bp, Mus musculus
cacccagaagtgtgctattctaccagctatttggtatctctagggcaacaaacttgacaatcaagatcagttaccacatgcctctctcctcgataaagagcaagctgcgaagggcgattacagctggacacaggtgagtgagccagaaggaactgaccagccttaggaaggaaccgtcacagctaggccatgctccagatattaatttctgagtcactggccaggagcgagatcccagtgtaagagtcccctgtgatgtaacgtctggaagaattccacagagaaagaagtcagaaacttctacacagctgacagatcaagatcaccagggctgggaagttttctcaggagtaaagtgtgaggagcggagtttaaatcccagcagccgtgtaaaagctgctgcaggagcatgtctgcaatctcaggcctgggggaagtgagcaaagacagcagctgctgagaacttactgggcagccgtggagacaaagtgccaagctccagattccgtgggagccccaggaaataaggtgaaagaacaagatgtagaaagaactctcagctccctcagcatcctgtctgcctgccttcaagctttcgtgcttcccaccatgatgacaacggactgaacctcagaatctacaagtcagccccaattaaatgttgttttagtcatggtgtcttttcacagcaatgaaaattctaagatagaagttactaacaaggactagggtattgctgtgataggcctgaccatgcttttgtggattttgtaactttggatttggaaagcagtggaatgctttaagtgggacttaatgggacagcctagtaggaatagggaggacattggtgctaagggtgaactctgcgggtctggctcaagagatttcagaggagaataacttgagtatgtggcctggagactgcttttgtgatactttggtgaggaatgtaggtactttttgccctcgtttgaagtgtctgcctgaggctcaagtgaagagattcaaattaattgcaatgacagaagtctcagaaaaggggcaagtccaatcttcatcatcaggatatcttcaatcagcaaccagcaaaaaacagcaaaacagcaaatgcaacagcaggaaccagcaacagcaggaagagcagcagccactacctcctctggtgtttatatactgctctggtatttatattctctctcaagaattcctagaaatctactgtcttcagctggcaaatcacatccctgccagagcacgagacaaatcatagtcagctgctgtggacaatctgaagcagacccacatcccacccctggaattaaaacaaaaacacattcatagaacataactaggggaaaaaaaaccaatacagtgagctttgggatggccagctttcttacacggaggctcagagctccacagtaagagagggtggtgggagtggtgacaggcagaaagactgtcatctttcatgactgcctgcataagaagctaagccccagggatagaggctcagcctctttatgaaagaatagcatgtctctaaaagaacatgtttagagggaaagactattagagccatattttggaaaatataatgagacacaatatttttttgtggatacagttttataataagggtggtagcaggaaatgtctcctgaccaccacctgctatcctgatgagggaaccagctgctatttggatgcctgtggtaaataccacccactttgatgccatcactctgatagcccagactcacttacaaccccttcactggggaaagagggatttttggtattacagctgccatcattgcagccattattgcaggcattacaggggcaacaacagctactgttgcccagaccgccactatgagaggctgtcaacgctgtagtctctaagtcagctgaggtgctacaggcataggagctactaaatcaacatctttatgaggccatccacattttacaacaacagattgactggcagaagagctggctcttgttagggacatgtgtctcaatagcttaaagatacctagtcagacaaattcctattcctgaaggcaagaaaaccactcacttgcccaattggtggtagatataaccacgtccataaccaactttggaagcgggaaccagggctcctatgacagaaaagaagcaggcttctctctgcaattcataaacaccgacaggaatatgttgcttttcctaagcccttcgctgtctttccaagaagaatttttttgtccttaaccaaatgtgaaatgcacattttgagcacaaagaatcaaagctccaacagcaaactgacatgacatcctttacaggaagtaaaatatttaaagacagagtcagagtcttctattcccctccccccaggaagaaaccctttaatattcccattaaccttgatgtgaatcacagcgctccttcttccctgtaggcagataatcatgactcaccacagacccctgactgtgggcttcatgtaattctggcagcaaaggccttccccgggggagctgtgtgaggccattggctgtgaaatctttacaaagtccagggatggactaggagtaaccctggagcaagctttgcactgtgtatcaaaacaagactccacattggggcatccacactgaggccaccttgtgaactaggcctcaccttggataagaaagaaattgcattatttttactttcaaatgtgttaaagaataaattttacttaaatatattctatattcaatc

SEQ ID NO: 2 - DNA, 25970 bp, Homo sapiens
ccatgaccgggtttggatctaataagtgaccagtgcacggggtggctcctggaaccaaaacaatgcccggcacagtggggagtcttgtgtcctcccggaacccgggggcctccagagggaaagggtcagttaaggacgttgctggggcttctgaaggagtgcagtgaccccaacaaaaattaaagccctggagctagcagtgggggaaacaagagtggtttcctaggagctgcctccttgtgaataaaaatgagtcggagacagaaaaggttggatcggaaaggaaaagaaatgctgagtggcatccactgtgggagccagaggtggcggggaggggggcagggttggagccttcagtttcatcctcacctttgctcatcccagagcctaagtctccagatcgagagctggaaccagacgtgatttattttgtatgcatcatcttaagaagcagttattgtgtactttaaggccaaacatgaattaaaaaataagaggcttaagtctccttgttaaaaataggagatttctctcccctcctttttcttggagcatttacttcagaaaccttgtcattgtaaatactttcttctctctttgaaatgtatataaaccctttcgaagtctagattggcatttcccagctttataacctaggaatgtctttctcaaggatcaggaggccatctctttgcaatgtaagcattaagggaggtagtaaacctaggtcccagtttccgagggaagtgaggggcctaacctcagtgggcaccttgctccaagttgcaaaaccaccttctctcataaagataggggacgttggccaggcgcggtggctcacgtctgtaatcccagcacttcaggaggccgaggtgggtggatcatgaggtcaggaattcaagaccagcctggccaacatggtaaaactccgtctctactgaaaatacaaaaattagctgggaatgatggcatgtgcctgtaatcccagctactcgggaggctgaggtgggagaattgcttgaaccaggacctgggaggcagaggtagcagtgagctgagatcgagccactgcactccagcctgggcgacagagcaagactccgtctcaaaacacaaacaaacaaacaaacaaaaacaaaaacaaaaaagtggacgtttgttcttcttctgagtaaagccaattagctaacacaggcaaataattctgtctagtcctcttacttgacaactagttattgttgatcttgaaaacgtatgtatgggttatacctacttgactatataaagggatgagatttctttctgtctttgcaatctctcagcagattgcctgcaatgtgcgctacattctggtttaatgcttattcaattctcaaagtgtttttgttttctattacttttgtggggaggatttctgggttaggagaagatatagtttttggttatatttccccaacatatcttaccatatgtctggtctgtgcctggcctcctggactcaatccatatttggtaagtacgatgtatgtcaccttggctttttatagctaaagttctcttaaaggactcgaacatttcccaagagattatcttaaagcttggtgaagtaagaatcagatattattattttcattttgtggtggtcccagaaggtaaggggcctgtccaagctcatgtaatctggagaaaagcaaggaattgttaaatatttgtactgcaagacactgcatgcctgctgccatatctcccaccctgcaggaacccatgctacgtggatttccactctattataatattacagttttcttttctgtcatctggaatattcacttttccctcaaatctctgcttctgctctgtatcagcatatttgatccactcctaagtataatatgtgtgcaagacaaagggggacacatgtccacagaaagacctgttcattaatgttcacagcagttctattcacgatcgccccaacctggaagcaacccaaacgcctatcaacaggccagtggctaaaccagttgtgtcgtatccatacaatggaatactactcagccactcaaataaactacctatacatacattcaataacattagtgaatttcaaaagtgtcatgctgagcttaaaaaaagccagatacaaaagattgcacatgaatccatttatgtaaatgtatgtaaaagattaccggagtccatgtacgtaaaagactacatatgtgcccatttatgtaaaattctgtaacaggcaaactaaactatagtgatgggaagcagctcagttgttggccagggtgggagtgagagagactgactgcaaatggacacaagaacactatgggatggaagaaatgttctatctcttggttagggtggaggatgcacacatgtatcattcgtcaaatctcaaaatgtgcatataaattgggtgtcttagtctgttcaggctgctcccccaaaatatcacaaattgggtggcttattaactacagaaattgacttctcacagttctagaggctggaagtccaagatcagggtgccagcacgatcaggttctggtgagggccctcttgacaacttcttgctaagtcctctcatggtggaatagatagaggagctctctggggtctttttaacaagggcattaatcccacttacgaaagctccaccttcatgatctaatagcctcccaaagccctcacctgctaataccatcacactggggcttaggtttcaacatacgaattttggggggaacaaaagtagtccacagaaaagggcatagttgattgtatataagttatacttctgtaaagcttattcaacaaataattatacatttttgtctgtgcacaaaatttgaaataaaaaagaaaaacaataacaaaatgcagtttgctttctcagccaaggtgacctagttcacagatgacaggagagcgacctttctgcagagccctctgcgtgcatcacaagggttgcccctagcccatccccaatctctgaaaagatgcagacagagagctggtcataataacctgcagctgggagtgatgtgctcccagagttcttctcccgggaaagccggcattgtcaggagaatcacatgcagcccacagctggcgggctttgtgttgaatcatgatgcttttataggaaagaaagggctgtgattcacatgtgtcatcgttgatgggaatattaagggctcccttctcctgggggaaaaatagaaacctctctctttaaatattgcatttctttcaaaacaaaaagtcacttcagctcctccccactgattcatttgagagaaaaagctgtcaggccccgtatttggttaaggcaaatcctttctcagaacaagatagtgctgtgtgtaggaaaatcacctaactcttgtcagtgtttatcagatgtggagagaaaacctgcttctctctttgtcatgtgacccatcgctcctgctggcatcgtgccatcagctgaggggtgactgggtttcccacccacaacaagggttcaggtaggggccatgggcctgaattgtaattcagtgttttgaaacagatagcacagttactagaggctgggaaggaaggatgaagaaggattggtgaatgggtgcaaatatgcagttagataggaggaatacgttctaatgtttgatagcacaataggtgactgtagttaacaacaatgtattatgcatttcaaaatagctagtagagaagacttgaaatgctcccaacacaaagaaatgataaatactcaaggtgatggatttcccaaatgtcctgacttgatcattacacattgtgtatatgtttccaaataccttatgtaccctgtaaatatgtacaaataccatgtatcatttaaaaaatgaaaaatataaaatagacatcacgaagtcagcagatatcagaatgggatctgagagagattttaaaagaggcctttgtacacaaatcagatttctattcaccttaagtatgggcttccttgaagtcttaaggaaagaaaatgaaccagactagcatttttcagtccagaaatcacgggctggcaaatgatctaaattctctccagtaactccaggagcaagggagctgagaagaggattggagggcctgaggcagaggggactaatatggatatcaacagcctgaggagctgctactggggacaacttgaggcacaccaaggttaccctctaccctgccatcctcaagtccctcttggttttggaaacctcaagtctatgaagccgatgaagcctcctgaaccacctcagagtggttgggtttactttaacaaccaaacccatcgccctgtccccagggagcttaagacagaggccaaggtgattgaagaaaaaaaaggaaggaaatagaataaagacaagagtcagctcaaaagcttcccctttcctggaattaatggcagcctcaatttattctcatctgtattaaaaattgttaagtaattaatgtgcgcatctcttctgaagctgatgtcatgtaagacactaggaaaactgagacctagtccttactctcaaagagttcccggctcagtaggggaagctgctacagaaacccgatctcatcattcagtgttttagaaacagtaacagtgataacaggctggtggggtggagttgtgttgagaaaacagagacagcatccatggttccagacatcctgatgatgacaataataatagtttacatttattaaatgcttactgtgtaccagacattgtggtaggtttttatggcatatcttcccttttccttccagcaactctttagaggaagcagtatcatcatcttcagggtcaccttgatggcagagcccactctactctggtggccctgggggtaacaggcaacccttcaggaaggactgtcctttcaccaacaccacctatggctgacagccagcatggttcgtttctagctgtgcttggccgtgaagttcagctgatcctgctctagctatttggtctggtgttgagctttgagatggcccagggagagggaaagcagagacagaggttgagacctgagtaatgaaacggagctgtcagggaggtgggggtgggagtgagagatttgtggacaaaggcagtggttttgggcttgaattgtgtcctccccaaaagataagctgaaattctaaccgcctgactccctgcaggataagaccttatttaaaaataagattgttgcagatgactttaggtcatactggagtaagtgaggcttttaatccaatatagctggtgtcctcctaaaaagaggagaaagacccacggagagaagatgaccgcgtgagaatgaaagcagactggagtgatgcaacgacaagccaagggacacccagcattgcctgttgttgccagatgctaggagagaggcatggaacagattctccctcagaaggaaccaaccctgctgacaccttgatttcagatacctccagaactaggagggatcgatttctgttttaagtgcccagtttgtggccctaggatgctaatcataaacgctagcatgagaaagggcttggtgtgtctgaagaaagatcagaaggctggcatggctggcggtgctgagtgagaagatgggagggcctccatgacccctctggctgggctgtgggccgctgtggccagcgtgcctgtcactgccccctggtggccaccgtgggatactccgagctgcacactggccttgggccaaagaaagaacgttcagattgagttaagagagctgggttgagcctacccctcaatctatttctagttaggtaactcgcattgccactttgctcctctgtcacataggaggatgattatgacatttggcccatccatctctcagggtgtgagtggggagcaggggaaactgtgtttggatgcttattgtgaatcatttctaccaggatgttaggtgcaatttctcctgttactccacagtttcagcagagacccagagggaaagaaaaatagctgcttatctatgcaggactcattaaacgattataaaatccattttgagacttttaggtcttggtccttacctaccgggcgactacacaagaagaggctggggggacagagcaagggacgggcagaggggagttctttagaatgagagctgttggagggataaggcaggagcatctccctgtgccctcttcctgtccccaccaccaccccacaacaagcctcgtgagattttgaggcttcaaaacaccccgtagagagcttgacaggtgcactggggagttgcagggacaaagggactcatacttgagccacatttcaaaccctaaaggccgagcctggttagagtggcctatggtggcaggatgaagagagggggcatgcagtgagctgcactcatagcccaggatgcaggagggtgcttcccacggctggatgaggaccccacagaagagagcaggcactgagttaacttgaatgggactctgccccaaaggaccatctgcaaggcaaggtgatcccagcagagtaactcaggggggagtggacttcagaagccaggggctggggaagtcatacatgacaaagcttgaggaacatcatcggggacctccaaggaacctacaggagtccacatgggagagagtgagcatggggtgtgtcacgcccagggcactgattccagaaaactcctgcagtcatcaaaaactgcactggagctttctgctccttatccacatccccaaagttggaggtgccaggaaagtatttgagcaagataaggaaatggagagaagtccccagtcttctccttccctgtgttgagtttttgagcccatatcggttctgagttttaaagtggatgtgggattatagctttggtagtcgaccaaactatctcagagtagactgaactatcccacagttttatggggcctcattctgccctattctggactttccagtatctgaaaaagaagacccttcattaataaaagggatgactggaaaagctatgggacctttctgagaattcatccaggcgcagggaaaaataatatgagagaagttgctcaaagaggtagaggtcagaaagagtcaagactttttctgttggtaccatttggaatattactctctcaacaaagaagtgtggttaggtaaatgttagccttggaaccttctttcccagaaataaaaggatcaacagggcccactctccttattgtatagcccagtgaatatttacttaagggaactagcccagtgagtatttacttaagggaaatgttttaaacagttatcaaatatggagtattagaagccattgttctctttcattaattttatttagattgaatcctgatttagcaataaaaatgcacgtctccacttaagaaatgctatttgccttcaaagaaggaaatgagactttcttaaattgtgctttgctatgatgttgttgaattgagatgattggaagagattatgattaagatatcagtgtcatatgtcatataggtttgtaggcactttgaaatgaataggaatgcattaagtcataaaatattattagaacagctgtatagtttacaaacaatctgatgcatactgagtaccacaaattctggtgcttgagtctcatacatactgctgttggcatttgtcagaaattctttggctaagagtaaaaggaacccaatttaaattagcttgagctgaaaaagaatacattggttcatataattgggaagtgtctaggggtgaatttgctttcagagatggctgaatatagggctgcctctctgtgtctcagctctgctatttatgtttcccttcatttcaacaaactttcctcttaggtgggcaagatgtctcttggtcctttctgactcccatgttcccttcttcccagctcagcaaacacagtagaagggaggcttgtttcagcagagtccacagagctataaatcccagaggcaattccacccctgaaccaacctctttggccaagagatgggaagcccttattggccagcttgggtgatgtggccatctctgcagtgggaagctggtgcacaatgatctacagtcctattaggacctcacagaatgggcaaagatccctaaggggaaccagcagaaggggatggaatagagtgctacctcagccaatacaagcccatttttccctttattcacagaattggcactttcttgcctggaattggaattgaaatggcatcccttcactgtgagccactattcttcccatccactgagttagtctgcctgtctgagaccaaatcatgcaactactggcctagagaaagaaactacttcgagtatgtgtagttttctccccaaagcaccagggttttattttttaacccatctatccatttgtttattaattatgtgcatacatttgcatttggagaggattcaaggcatcttataacagcacaaatatgatataatcagtaagatgagggctaaaaacagcaaataccaagtaagaagggcatcagggagatgattgtctaaaagagtctgagctaaacatggcaacttgcagttctgcttttgatactggcaaaagaagctctcgttagaccaaccctcccacaaattgtacctataaactctggacaatcaatacttggaagatggaaatagcatggggtgagttgttttttttttttgttttcatggcttctcagctaaaggcaggcctcattgtaccacatggcagatagctaaaatgccaatccaaaagctacaatccttctggcctgaagaatcagatgataacattcagggcaaccacaactggaaagtgaaagaggggaaatccagacatggaaagagccagagagccccaaattccttatattttggccgactctctgactgaccattaaatcatgcatacatagtgcaaatttcaagcaacccagtgaactgagatttgagccgcaccctgagaggcagagtttgcagttggaatccaaccaagttaattccctgctaaaagaaaatcaaccttcactggaggagtatggcagaatccagactctgcacagcccagcattcacaatgtccatgataccatcccaaataagtcaacacactaagaaataggaaaatgtgccacattctcaagaaaaaaagataattagtggagattgatttggatgtgacccagatgttggaatgagcagactcataaaagcttaaaataattttcaaagcaggtattataattgcgctaaatgatacaaaagaaaatatgttgtgatgacttacaaaagaggaaatctcagcagagaaatagaacaataaaaagaaccaaatgaaaattatagaactgaaacaaatatatacttgaaataaattaagtcattaatgggcttcatagcaaaatggagatgaaaagagggaaaagtctgaatttaaagatagtctagtaaaagttatcctatctgaaggtaggtactttcacttagcaaagtgtagccctaagtttactgtccaccaaagcacagagggaaaccaggtgggttgtcaactgagttgctcacctgaagagttgtgacattatgtttaaggggctctaacagccagcactcgcatatcctgagacacaccttgggccaattactaatctcttttccctccactccaacaccctcctattctctgctctgtgatgctggggtggatattctgcaaactgcatttcttttttgccagctaattctttgttaggttctggcacttgggcaatagagggaaactggaagactgaaggacagagaagggacctcctttctatttgtttgtcactctgtttgcatttcccctgtagcccccttccctacagctgtaagtggtctggtctccggtttgttccaacacgctccctccagaggtaccagcacctgctgggcagtgcagtgtcttcttcttagaggtctgttgtgccatttgtatgggtccgttttttaagcatttaatttctgacaacttcaaactcttttctttgttcccgcaaccttagaaatgatagctgcttcctgcatttacgatctctgtgtaatctcagtttttcttttgaaattttcagtcctctaattcttattttaaattttctgttaaaacaactcctggggtttctattttcctgacttacccccaactgatctatatccctttttattgactgatgtagatctctgattaccatatgtagggctggcagtggaaaaaattgtgaagaacgaagtttgttcttggatccaagatacctcctttcagagctctgatgcccagatatccaccacaattatgcaatgtcccaggctcctgccagccctagaccccttccattccatcactcatacccctagggactgcattctgataggtagcaataagtgatcatggattttggtgagataaactagttccctaatatgcaagctaaagtgtttccctcccatataacctacttccttggcttagataaacatacttcttagaaaggccttaatggaatgtcaaaaatggaggaaatctcagatgtagtagctaacccatggttttcatgcagtagacatagaaatagccatctcctctgttcacgtgtagaaacatgagtgcttggatattccttcttcttttgacacctttttatctgggccactgggaaagttaaaggagctcagccatgaaaaaagaaacaaaacaaaacaaaacaacaacaacaaaaacaaaccctagagtttcacagacactatttgttagaaatacagcaaattatttttagctggttacagataaattagtttaattttctcaagtagtcagttgaaataaaatgttctaaactttgctttgagtaattgaatgtaatttgaattaggggttgtgtgtaactcttgtatgtttgataatcaacaactatccccacttctttctgtagaaaagaaaggattttattttctgtgactttcagatcacatgccttatgtatatttccatgctcagaaatatcctcctaaccctgcaatttaaccaacaaatccaatgctttaagatacttcgagataatacaagcaagtcacaaggcatatggctcagtaaatggtaactataataacactgtgtcctattgtattttccaaaaatggtcttaacaatcttttcctccccacatattcttttgaaaccttgccacatctccatcaagaaggggaatctatgtcccctccccttgaaactggatgggcttttgtgactgtcttccctggtagagcaggaagaaagtgatatctgagacttctgatgctagggcatcgaaggtgatattgtgcctgccttcgcgctggacagtcttcattgcaaaatgacatagagatagagagggttgctggaggaaccggtggttccagccatcagctatttgaggcttctcatcctaggtgcagacacaccagtgacccttcagagaattctagccccagtcaccatctgaccgtacctgcatgagagacagaacccctagactcatgaggtaataataaattattgttattgtttgaagccatgaagttttgggctgatttgttatgcagcactggctaatggatacaatatttaatgtttggcaggtattgatgatgagtcacttactatgctaaatgctttgcagggattacctcatttaattctgattatcccagagataagcatttttatccaccctgagagaagttaagtgatctgtccaatgtcacacaacagtcaatctgatatttcaatccagacagtcagactctagagctccgagcatgcaaatactaagccctaccacatgtagcgctccccatattcactgcttttctgctgggtaacagcatcccctctgaccctggcacacccattgctctgtcctccagcagcacagaagcatggtgggtgagcagacaggtgccagcaaggccatatggatccagccaatgctgaaccgcaatattatcattttattcctttggctttaagactatgtgttgtgttttttaactggtggtgacagtggtggatggagacatgctatgtcatattgcccgtttgcacttgatgagtttgtaggatgtcttcctattggaaggttttaatgacatctggggacaggagagggaaagaaaaagaaagaaagaagaaaaaagagagggagggaaggagggagggagggaaggagggaggaaggaaggaaggaaggaaggaaggaaggaaggaaggaaaatagtgaaaacatggaacagacacccagaagggaaagggtttggaggaggtcgaggtgccagaggcaagcattcatttgtgtaagattcattcattcattcattcattcattcatcgtcattcaaacattgtgttgaatgtccactgtatgctgtgccctgcgtggaatgctaatgatggatctaccctctagcaaaaggtaagtcctaccctagttcattcgtacaaatagaggtgcacacaatgtatcatgggagcacttattcctcaacaggcagatggatcagaaaaggattcctagaagaggcgacagctgagctgaggcttaaaggataacacagttaaccaggccacaggtggtgagtgtcaggccaggaaacagcacgagcaagggcatgggatacggcgcatgggagcaggagcacttttgagctgttggaggtaaaatgcaagttaggaagcactgagagatgagcctggcttagaggtggcagcgagggccttggtggtcattgtcagagttggagttttgtcttgtgagtgatgcggagccactgaagggtcttcaggggactgtgagagaccatacatcaccaggtcttcctctttcttctgccaaactctgcccaggaggatttggtgtctaaatcctgcacattgaagaccaacccttaaaatgaaatttgttcaaattccagtggcaaaatcagagaggaaaatgttctatcagagagaggaaatatttcccaccatgtctgcagcccttaatccgtgaggtcaacatgcccagctgcatggtcttctggcagaagtgttattgcagtcatccacagcatgcttctaggggcaggacttaattgaatttaacagcagctaaatgctcagctaaaaagagaagttatgggctgggtgcagtggctcacgcctgtaatcccagcactttgggaggccgaggtgggcgggtaatgaggtcaggagttcgagaccagcctggccaacatagtgaaaccccatctttactaaaaatacaaaaaattagctaggcatggtggcgggcacctgtaattccagctactcgggaggctgaggcaggagaatcgcttgaacccaggaggcggagattgtagtgagccgagatcatgccattgcactccagcccgggtgacagtgcaggattctgtcaaaaaaaaaaaaaagagaagttatggaaaactgtgatgctgcagtgaaatgaaatcaggctgctatgtgtcaggctgcgatgtggggccctgtgtcctgagtcacacaccaaaacaggaaatcccctattctcatcttgaaaaatttattagtaaaataaagtaaataagaccaagaaaaataaggctgctattatttgaatcatttagtgaaaaagaaaagctttctagaaccaacagaacatggtaatgaacactctgcccccatacttatcagtctgtattggcattttctgtgtaacatacctttctttcctactagaaacagtttctctagttcaggatctatgtcttatttatcaactcccaaaactgtcccttctttgaattcctttgatacttaatacccaagtcactcatttggcatttatgttaccttgtatggtctattgtatttctattctgtttatacatagtgactcttgaactagactataaactccttcgaggtagaagtcttgctttgaacattttgtgtccccatttgttatttgttgaaaacaaggcatgccaaggaaggtgctaagggctggatatgggataagagaccctggtctacctttggggattcacaatctggggaatttcctcaggcagggcacaaaacctatgagacaatcctgctttcctgctgaacttggacctgggacagggtggggatgggtgaggctggagctgctgcagcagcgtgcctccaatgcctataaatttcctgttggctctcatccgtctgggctgaattttctatcaaccaatagaaaatacagacaggtattatgaaagagggtgttaggaaatcccatgcaattattatacatgtcacaaatgtgtgggtgcatatgcacctgtgagtgtgtgacagacagagagggaggaagagcactaggaggaaatgccatctgaactgaatcttgaaagacaaacgggagttaacgaaagatgttcctgagagagtggcaggtctgtgcttgcatcctgatgtcagagtcagataaactccagaagcctcagggcctctgcactctatcccagtgcttccccaaactcctgttgagggcagactcttttagaatatgttaacgaatggatgtgcagaagaaaactatcttctgagaacaaaattgctctagtttctgcattgctggttttctctgaacctttaatatgctgataggcactcaacaacagaatcctttgttggcagagcacaaattaccctctgtctgttgaacagtttcagaaagtttatgaagcacttttcaatgaggtgtggagtaataaatgattcagcaacacttaagtcctgccaccctaggcctaactggttacactgtcatgaaaatttacaaattacatggtaccaaagacaatataaacacaatctattgggtaaacaattgcatgatggattactgtagacttatcaatagttatggcataattcagtctttatttttgcatatgaaaggctctcacgcaagcatttttctgtagctcattggaatacagtacagatggcctggatcagtgcaatgtgcaacaagaggactatatcgtctcagtaaaaaccttgaagagatgaacccatttcagagacccagcaagggatcacctgcatatacaggcatcagcagaaacaggaatttgggatttatcctggaggtagtggaagcttttggaggtcttaaccaggtgaatgcaatgattgggtatattttagaaagctcattcacagggagagaggacttgaagcggggttgttactagagacaaagaaactagttaggaagttatttcaggtaatgagggagacataatttaggggctaaaccaaggaagtgacaataggaatggagagaaggcacagcttgcgaagcatttaggtggtagaactgattgactcagctctgaggcagctgtgggggtgagaagctggggtgggtgagggtgggaagagggtggggagggttggaggagtcaagaatgaaccatgacttctggttcgtgcaacagagtgaagagctgaggccccatggtttccaggcagcctgtgccagtgagagaaatgcagaatagagtttgatttgaactctggctctgtcaacgacttggtactatggccttgtgcaagcaagttaacgtctccaagccttacttttatcttttgtaaaatgggacgatgataccccgctcttgcatgtgtgaaataactaacacatagtctgtgcttcagaagtgaagtcactgctactcagagaacacagaaggagaagcagtttacagagaaagatgagtttcatttcagaaacactgcacttgaggtggcttggacagccatgtggaggtgtccagtaaggcattagatatataggtatggaactcaggagaaagatctgggctggagagagttctaggggccactgactttttgatattaggccaaggcaaggaggtaaatgagaacaaccagagagagtgtggagagggagagaaatgcaggccctaacggacggccttgggagcatcaccattcaacagctgaggaaaggaagcagagccggcaagggagatgttctggagcctgcagggctgtgcagtcagaaggaggcgcaaaccagaatctgaagacgcaggtttgagtctcagctcccttgctgactagctgtgtgatcctcgcaattcaggtaacctctgtaagcttgtcttctcattcgtcaaatgggaatgctgctgataatgtctacttcctggagactttggtgagctaaggtgtataatttttttttttaagctagagagccttataagtggttccatgtggtcattttttccccctagactgaatcggcttacatctaaaagcaaggtcactttaagagggtctctgtgactctggccttgcttgctcagcaggaattccagcctgctcaaattcctctaggcagctctgggatccctgggcaggaggtacagtacctgatgcctgagtgccccaaaccccttccgaggaaacggggccttaagcacaggccctcacgttgtcccacttgacttcaccaccctggctacacctggtgggaattgggatgggccctgttcccaggggagctgaccagctgaccagccgaccagccagcagctccccacgtctgcccggggtgagacaggagaatacggtctggaggcagggaacataagactgattcatgctgacttcctagaactaaatcaaatggaaatacttcagctattacaggaaatatcctcaccattgacacaaggcgtaaacccagtaaatgtctttgtaactttacttgatcctcttcatttacacagggcgtacaccaagtaaccaatggaaacctctagagggtatttaaaccccagaaaattctgtaacagggctcttgagcccctatgctcggggccactcccacctcattttcaataaatctctgcttttgttgcttcattctttccttcctttgtttgtaactttgtccagttctttgttcaagaccccaagaacctggacaccctccactggtagcaagggggccatgtgatagagatcctgcccctctcctgtatggacatttggactcagcaagcgaggccagagtcacagagaccctcttaaaatgaccttgcttttagatgcaggctgattagttgccaaagcccagcaactggcaggatgggagacatggtgacagtgtctgggtggctcatggagcccctcacagccatgggtggtggcaggaaatgggcagaggtgcctggtgcccagggtggggctactctccacctgcctttgtcaggctgcttcccagtttcttttttttggtctgtgttcttgtgtgtctcacaatgcactccatttgttaaagtaacttgagaacgtggccatgtcttgtggcttagaggaggctaattgtcacgggagactctcacgatctctcaggagggagtggagttttctcagaataaagagcatcagctacagtgaagaatggaaatggggctaatgtctattgactggggactggtgagcaagatcatggcacctccgcacacgatgaaatcctgcagctggaaaacagaacaaggaagctctttgtgtactgataaggagtgatctgcaagatattttcctaggtgaaaaatgtgagcttcagaatggtgtagcaaaatgaaactcagaaaatatctctagaatattacacctcatctaatgacgctggttgcctatgaggagggaagctgggggcctgggggcagtgataggagaagaaattttcactaagcattgtgtaataattttcctaaatcttgaatcatgaaattcagtcacctctttaaacaatcaaatcagaaaaacatctattaagaacatgacctataaagttgagcaagtcctatgcttgggcttgcccccacaatttattgctgggtaaagttgagcaagttactcaacctctctgaactcattttgctcatctgtaacataatcacaggcttgcctccacaatttattgctgggtaaagttgagcaagttactcaacctctctgaagtcattttgctcatctgtaacataatcacaggaataatacctgcctgatagggctgattggttattgttgttgttttgttttctttctttctttttttcattttttttttttttttgagacggagtctcactctgttgcccaggctggagtgcagtggcacaatcttggctcactgcagactccgcctctcgggttcaagcgattctgttgcctcagcctccagagtagctgggactacaggcgtgtgccaccgcgcccagctgatttttgtattttttagtagagacggggtttcaccgtgttggccaggctggtcttgaactccagacctcaagtaatccgcctgcctcagcctcccaaagtgctcagattacagctgtgtgccaccacacttggctgttgttactggttttcacacaaggcattttccatttcccattttcctcttagcctaatagcatgctccttaagctacaaaactgcaaagcagtgctatcctttgtttgcagttttcctttttctgttgggtgctctccataagataagggccattgaggttgggccaggtaaattaaaaccagcctctactgtactcaaaattatctcttctatttggaatgggctcattgtctttggacccagcccatgcctgacgctctctttctttaaaaagagttcttttcctagccccttcttggtccccactgcaaagttttctatggattacataagcacttctagattcccggtgaacacagtttaccttaagctattaattaaaacattcgttattattacggtgcccatttcccattatcttcaatcacagtattttgtgcattgtagtcactgaagtgtgaccaacatttcattcccgcatagtgatgcctccagggctcgtggactggacagtctagggtcgtgctctgctctgcaggagacagggagcacccaagccaagttggttcctaccttcccggcactagggggtgctgtcttgtttagctattttcttagttttatttttgcctttgaatagatgtgcctgctgcacttacatctccctgcaaggatagcaaaagctataggaagatgattgtggaaaggaaaatagggtctgtgcaacgagctgtctggcttcctctctgtggggaatgagcattaagacattcccaggctcagtaggtctggaatgggaccgagatatgtgcattctagtaactattcaggtgatattcaaagccacagatgggtcttttatttcattcgtgatttgcttttcccatcttttctttaaaatgtgtctgccgactctcactcatggtggtattgcccctcctgggcctgctgatctctgactgtgtatttattagatcttcatctgtgggagtcttctcggcttaggttgggggtgctctctttagggaagatttgcatctgcttcttctgagagccatgggtcactgccagcttggaaccatttagagctcctcgtgagttctggtttaatgaaggtcttaggtttggcctcccctcctcattgctggcccaagagtgaggatctggatcatttgctcttagggtagttctgctcctccctgcttgctcactgcttccagctctaatttctacttatagtttgctccatttcatgtggaggagtcccttggcaacatcctctatctcctacaagcccatctcatctatgttttatctaggatgtaattgttttgcagaaggagagctgtattagtccgttctcatgctactaataaggacatacctgagactgggtaatgtataaaggagagaagtgtaattgactcatagctccacagggctgaggaagcctcaggaaacttataatcctggtggaaggggaaacaagcacatccttcttcacatggcggcagcaaggagaagtgccgagcaaaagggggaaaagttccttataaaaccatcagatcttgtgagaactcactcactgtcaggagaacagcatgagggtaaccgcctccatgattcaattacctcccaccgggtctcatatgtggggattatgggattacaattcaagatgagatttaggtggggacacagccaaaccatatcaagggcctatgagaatatttggtctgagatgttatcagtggaagtctttttttttttaatgcagatgttcttatttctatgagacaaaatggtaatacatctattgtgagggaattattcatcaaacatatgcagttttatcattctgcactgtaggaaggttgaaggaacacgtgtctgggtgtagtttgtgtgttacttctcacagaattactggtgtattatattatgttctattctgggtgtcttgaggcctgataagacaagcatggccatgagaaattctataattccactcgtgtctccacctacctttttcttcccttttgtccagtaaaggaggtgtctctgttgcatcaaagcccaccccacacagtgggttctagatcccgtccccttcatcttctcaaggaccctgctccattggaatcctccctctgctgcaccatcaactccctcgcagggagacagttcccatcccacattttcccatctttaaaaaacccacccttggccaggtttggtggttcacacctgtaatcccaagactttgggaggctgagacaggaggatcacttgagcccaggagttcaagaccaacctgggcaacatggtgaaaccccatctttacaaaaaataaaaaattagctgggcatggtggcacccagagagccaggggtcactgccagcttggaaccatttagagctcctaatgagttctggtttaatgaaggtcttagtttcggcctctccccctcgctgctggcccaagagtgaggatctcgatcgtggcacatctaccataatttactcttagggtagttctgctcctctctgcttgcttactgcttccaggtgcttgggaggctgaggtgggaggatcacttcagcccaggtggtcaaagctatagtgagccatgatcatactactgcactccagcctggctgacagagcaagaccctgtcttaaaacaaaacaaaacaaaacaaaacaaaacaaaacaaaacccacccttgaccccacctaccctaattcacttttctattcctcttaggaaccagatctctcaaaatatatgtctatagctgccactttccctttttcactctccatttgcctttgcttttacttggtttctatttttagcaaggttatttgcacatattttaaagactcaagagttctataagacttgttatgaaaaccaacagtcctctgctttccccatcagagacaactacttacaactcttgtagctgctttttctttttggcatttagctctgtgtttctgaataaaatgctagtattgggctacttaatgatccctcaattgtgagcagtagctattgactgcccctacagaggaggaggatttagctctcattctttccccttcccacagcatgcacatacattctttccacctcctaccattcctgcatggttacatcttaactttccttggttcagtaatccatgtttactttattttgattatatagtgatatcgagagcagagtcatctagtaagcttagactactttttcttccttacacaacttttaattttccttgggaataattttttatttgcttagttttctacttattaccagtccaacttcaaactgccataatttatctttctgctcaaaacgtctagatttatgctattagttttgtcctcttgaagaatcccttccaaaacctctgactttctccaatttggattgtctccaaaccaccctcaagacctggtgtagcactgttttgttttgttgtgttttctttttcttttaacttttattttaggttcaggggtacatgtgcagatttgttatatgggtaaacttgtgtcatggaagtctggtgtacagattatttagtcacccaaatactaagcctagtacctgatagttattttttctgctcctctccctcctcccactctccaccctcaggtagacccccgtgtctgttgctaccctctttgtgtccatgaattcttatcatttagctcatacttataagtgagaacatatgatatttggttttctgttcctgtgttagtttgcaaaggatagtggcctccagccccatccatgttactgcaaaggacatgatctcactctttaaaatggctgcatttaatggtgtgtatgtaccacattttctgtattcgagctgccgttgatgggcatttaggttgatcctatgtctttgttattatgaatagaacagtttctttggaggtcttttcatcagtctagggattcccttcatcttttgcctgggtcctggctccaaacttttctttcctgttttggtggaatgcgtcttctagttacttcttcagaagtgcatgtgaggtaaaatttttgagaccttgcaaatataaatgcctttattttgacttttcacttaattgatggctaggaataaaaaatctaggcttaaaataatttattcagaattttgaaggctacagtccactgtattctagcctctagtcttgtgattgagaattccaaagacattctgattcttttaaaacttctgttttttatttttagatacagggtcttgctatgctacccaggttggatttgaactcctaggctcaagggatcctcccaccgtagctttccaagtagctgggatgatagacacttgtcaccattcctatctggcattctgattcttaatcttatgtatgtgacacatttttctcttggaagcttgtaggatcttctctttgtccccaggtttctgaaaatttatgatggcattctttggggtacgtttaatccgttttgacagtttaattctagaaaaatttcttgaattatttcattggtgatttcttcccttctgtgctctcttatctctttctggaatccctattatttgggtgctggatctcctagactgatcctctaattttcttttctatattctgtttttttttttttttttggccttttcatgcctcagtttcgtttttctagctctctactaagtttttcacttcttccatcatatatttaattaccaagggctttttgtggttgttgattgttctctaaaggttctattttatttgtattcttttctttttttagggttgtaacatctcctctgatctctctgaggacattaaatacagttttcttgacgttttcttcctcttatatagttcccatttttttccaagtagctcttttgcattagagtctttcttcagatatcttttaatacctgctgtctgcttatccttgactgtggaaaccaaaaaagctgatttgaagctttgttcacctgagtggaacttacccactatgagcttcacagtagggcaatctggcttgactatttccttgggcagccctgatgtatcgttaggtgttttctctggattggttggttttccaagagaatctttcctacgtgtgctctggcaggtgaggagggaaagaagcctgaaagtctcagcacaggtaagcatacagtctcttaattgtcctatttgatattccagtcaagagactcattgtttgatcttttccagagaatgaatccagtcttctgccaggatggggaagggcatctattaagagtatggtaggggtctgcaaatataattcctttttaaattgctttcaactaattctccttattttaatgccaattcccacccacttgccctacctctatttttaaagtttccggtgccactaactcctgagactttgaaaaatttgaggtgcaaatggtgttggttttaagtttcctcactactgacataggattccaccttcttcagcccactaaattagctaaaacttctccaaatggtttccagctttcaaaatactgtggcatttttgtcctctccccgtcttaccatcctcatgggtttatgttaaagataatcactttagtgtttgtgtgtgtgcgtgtttggcttgagaaacaagtaaaattacatgcatgtgttcccatctactcattaacccacttgctctggctgtgaagctacccctccactgccacttttaccaaattcaccagtgatgttcatgttcccaacaatgggctgtttttcatccttgactcacacaaacactcaggagccttagtcaaaattgaatgctttcttcctgacaggtgctcttctctgggcttttgtgacactatacctttttgggtttgttttctgcttctttgatttctcctccccagcttacttctcaagttcattttcctctgccagtcctctaaagaggggtttcctcaaagcgtgatctcaggccatcctctttctctctctctatgtctaccactttctccttaggaaattgcacactttgttcatggctccaacactgatcttcttttgattcttcatcttctctatctaattgcctacttgtctcccaggcatgtttaactcaaaatatccaaaacagacatcctgatttttttttctccaaatttattcctcattaagtcttcctcacctacctagatatacaagcagaaactggaaagtcattcttgttttctccctcttccttacttctgacatttaaaatatcagcaaggcttcttactctacctccagaatgtgtcttgaatctatccattttctccatcatcactgccactgccttagtgtatgaatcactattatctcttgccttgactattgtaacaatctccaaactgatttccctgctttttctctgggctccccaaatcagttctccatacgagtgatcttttatttcttttcttctctatctcttttttttttttttttgagacagtctcactttgtcacccaggctgcagtgcagtgttgtgatctcagctcactgcagcttccacctcctgggttcaagtgattctcctgcctcagcctcccgggcaactgggattacaggtgcatgtcaccatgcccagcaattttttgtttttgtttttgtatttttagtacagacagggtttcaccatgttagctagattggtcttgaactcctgacctcaagtgatccacccacctcagtctcccaaagtgctgggattataggcatgagtcatcgctcccagccacaagtgatatttcaaaaacagaaaatcaaattattctctcttttaatgaaaatacttttcattgcccctaaaacaaaatccaaacttgttaccatgttttctgcaccagacttgcttttcccactagggccccttgttaattccctggatgctaccacttctgggtactagtctcctgctaggacatggagctctagaggtgccaaagcacatctctctcccacttttgtacatgttgcttctggtcagaagagtgccaaatgagtggaatctatccctgaacatcaaagagctttgagtgaaattattttccaagtttcacttatagcctcctcctttctataggatttggccacaggttcacggaggaagggaatttggggagccgaggaatgagcggggcaggaagcacatacgaacgtcctgcaccaggctgcactaagaaccttccctctgtttgatggctaaggccttgctttgtgagcaagcttgctgtcctaaaaccttcctcccggaaggattctcagtctgttaagaaaatagaggccacccaaacctttgtcaagatatgtttagaaatatttgaataatttagctt

SEQ ID NO: 3 - DNA, 22 bp, Mus musculus
cctttaatattcccattaacct

SEQ ID NO: 4 - DNA, 100 bp, Mus musculus
gatctcgcgttgacagtgagcgcgcctctctcctcgataaagagtagtgaagccacagatgtactctttatcgaggagagaggcatgcctactgcctcga

SEQ ID NO: 5 - DNA, 100 bp, Mus musculus
gatctcgcgttgacagtgagcgataggccatgctccagatattatagtgaagccacagatgtataatatctggagcatggcctagtgcctactgcctcga

SEQ ID NO: 6 - DNA, 100 bp, Mus musculus
gatctcgcgttgacagtgagcgcttggtgaggaatgtaggtacttagtgaagccacagatgtaagtacctacattcctcaccaaatgcctactgcctcga

(19) United States
(12) Patent Application Publication
(10) Pub. No.: US 2012/0183511 A1
(43) Pub. Date: Jul. 19, 2012

(54) INDUCTION OF NEURONAL DIFFERENTIATION IN NON-NEURONAL CELLS USING A NUCLEIC ACID MOLECULE

(76) Inventors: Saba Valadkhan, Cleveland, OH (US); Fereshteh Jahaniani Kenari, Santa Cruz, CA (US)

(21) Appl. No.: 13/395,984

(22) PCT Filed: Sep. 16, 2010
(86) PCT No.: PCT/US10/49128

§ 371 Date: Mar. 14, 2012

Related U.S. Application Data
(60) Provisional application No. 61/242,995, filed on Sep. 16, 2009.

(51) Int. Cl. A61K 35/30; A61P 25/00; C12N 15/85

(52) U.S. Cl. 424/93.21; 435/455

(57) ABSTRACT

A method of transdifferentiating a non-neuronal mammalian cell into a neuronal cell including transfecting the non-neuronal non-terminally differentiated mammalian cell with a nucleic acid that promotes the transdifferentiation of the cell into a neuronal cell, wherein the nucleic acid is substantially homologous to BORG RNA.

### RELATED APPLICATION

This application claims priority from U.S. Provisional Application No. 61/242,995, filed Sep. 16, 2009, the subject matter, which is incorporated herein by reference in its entirety.

### TECHNICAL FIELD

This application relates to methods and compositions for inducing transdifferentiation of a non-neuronal mammalian cells into a cell of neural lineage, and also to compositions and methods for treating neurological disorders.

### BACKGROUND

Many human diseases are caused by the loss of neurons. The loss of neurons is exacerbated by the inability to generate new neurons to replace them. Despite intense research, generation of neurons from other cell types has proven to be extremely difficult and remains a major limiting factor in both research and therapeutic efforts in the field of neuroscience.

In vivo, differentiated cell types vary in their ability to undergo proliferation and continue cycling upon physiological demand. Neurons, are known to be terminally arrested in the G0 phase of the cell cycle and do not proliferate after birth. Other cell types, however, have been known to have high regeneration ability that is retained during the post-natal period. They include muscle cells (skeletal myoblasts), several connective tissue cell types (cartilage, bone, and fibroblasts), liver cells, epithelia (skin and gut); hematopoietic cells (bone marrow and spleen). Not only can these cells regenerate themselves but can also generate cells of distinctly different phenotypes.

States of terminal cell differentiation are often considered fixed but in some cases, they can inter-convert. The conversion of cells, including stem cells, in postnatal life from one cell type to another is termed metaplasia. The conversion of one cell type to another usually arises in situations of chronic tissue damage and associated regeneration. Some changes may be indirect, occurring through an intervening stem cell, whereas others may be direct transformations, sometimes called transdifferentiations.

Despite the advances in stem cell biology, obtaining stem cells from adults and differentiating them into neuronal lineages both represent significant challenges, if not impossibilities. For example, adult stem cells can be obtained from bone marrow, followed by laborious separation procedures and finally induction of neuronal differentiation by the addition of several chemical at exact concentrations, which is not possible inside living organisms. Thus, a simple, easy to use method for transdifferentiating non-neuronal cells, which are easily available from patients, into neurons is highly desirable for use in the fields of both health care and neuroscience research.

### SUMMARY

This application relates to methods and compositions of promoting transdifferentiation of a non-neuronal mammalian cell into a cell of neural lineage. The application also relates to the administration of the transdifferentiated neuronal cell to a subject as part of a cell therapy that calls for implantation of a neuronal cell.

In an aspect of the application, a method of transdifferentiating a non-neuronal mammalian cell into a neuronal cell includes transfecting the non-neuronal mammalian cell with a nucleic acid that promotes the transdifferentiation of the non-neuronal mammalian cell into a neuronal cell. The nucleic acid is substantially homologous to mammalian BORG RNA.

In certain aspects, the transdifferentiated neuronal cell expresses at least one neural specific antigen selected from the group consisting of MAP2, TUJ-1, NF200, Tau, synapsin, and nestin.

In some aspects, the non-neuronal cell is transfected with a vector comprising BORG cDNA. The vector upregulates the expression of BORG RNA in the non-neuronal cell. In some aspects, the nucleic acid encodes functioning non-coding BORG RNA. The vector can also include a promoter operatively linked to the BORG cDNA. In some aspects, the promoter is a CMV promoter.

In some aspects, the method further includes culturing the neuronal cell in a growth medium. The growth medium can include a low serum cell culture medium.

In some aspects, the non-neuronal mammalian cell is a cell of mesodermic origin. The cell of mesodermic origin can include, for example, a fibroblast, myoblast, osteoblast, chondroblast, or adipoblast.

Another aspect of the application relates to a transdifferentiated mammalian cell that over expresses BORG RNA. The transdifferentiated mammalian cell can express at least one neural specific antigen selected from the group consisting of MAP2, TUJ-1, NF200, Tau, synapsin, and nestin. In some aspects, the transdifferentiated mammalian cell has one or more morphological, physiological or immunological feature(s) of a neuronal cell. The transdifferentiated mammalian cell can further display a lack of proliferation.

In some aspects, the mammalian cell can be transdifferentiated from a human non-neuronal cell. In other aspects, the non-neuronal mammalian cell is a cell of mesodermic origin. The cell of mesodermic origin can include a fibroblast, myoblast, osteoblast, chondroblast, or an adipoblast.

Another aspect of the application relates to a method of treating a neurological disorder in a subject. The method includes obtaining a sample of non-neuronal cells. The non-neuronal cells are transfected with one or more nucleic acid construct(s). The nucleic acid construct(s) include a promoter sequence operatively linked to a nucleic acid substantially homologous to BORG RNA. The non-neuronal cells are transdifferentiated by the BORG RNA into neuronal cells. A therapeutically effective amount of the transdifferentiated cells can then be administered to the subject to treat the neurological disorder.

In some aspects, the non-neuronal cells are obtained from the subject being treated. In other aspects, the non-neuronal cells are cells of mesodermic origin. The cells of mesodermic origin can include, for example, fibroblasts, myoblasts, osteoblasts, chondroblasts, or adipoblasts.

The transdifferentiated cells of the application can be administered to the central nervous system of the subject. The transdifferentiated cells can also be administered to a lesion site of the subject.

The neurological disorder can include a peripheral nervous system disease, a central nervous system disease, or a neurodegenerative disease. In certain aspects, the neurological disorder is selected from the group consisting of Alzheimer's disease, Parkinson's disease, Huntington's disease, amyotrophic lateral sclerosis, elderly dementia, Tay-Sach's disease, Sandhoffs disease, Hurler's syndrome, Krabbe's disease, birth-induced traumatic central nervous system injury, epilepsy, multiple sclerosis, trauma, tumor, stroke, and spinal cord injury.

### BRIEF DESCRIPTION OF THE DRAWINGS

The foregoing and other features of the present application will become apparent to those skilled in the art to which the present application relates upon reading the following description with reference to the accompanying drawings, in which:

FIG. 1 illustrates: (A) a schematic of the genomic locus of BORG in mouse; the location of introns and exons in BORG are shown by thin and thick lines, respectively and their size in nucleotides is indicated; (B) images showing in situ hybridization using FITC-conjugated BORG probe and mock-treated cells; (C) a chart showing tissue expression analysis on 2, 20 and 60 day old mice (shown with triangles, rectangles and diamonds, respectively) indicating the expression of BORG in neural tissues and kidneys; (D) a chart showing BORG is expressed in primary neurons, but not in primary oligodendrocytes or astrocytes, based on RT-PCR analyses; and (E) images showing in situ hybridization assays indicate the expression of BORG in primary neurons.

FIG. 2 illustrates: (A) images showing BORG overexpression or shRNA-mediated knock down in N2A cells; and (B) images showing vector-transfected and BORG overexpressing C2C12 cells before and after 5 days of incubation in differentiation media. The multinucleated myotubes are visible in control panels after differentiation. The bottom right panel shows the BORG overexpressing cells at higher cell density.

FIG. 3 illustrates: (A) a chart showing clustering of genes involved in various aspects of muscle differentiation and function and their global down-regulation in BORG overexpression cells; and (B) a chart showing BORG overexpression blocks the myogenic differentiation program at step of induction of MyoG expression by MyoD. Bars marked control and +BORG represent RT-PCR-based expression levels of the genes indicated on top in vector transfected and BORG overexpressing cells, respectively. For each series, the expression level in +BORG bars was normalized to the level of expression in control which was set to an arbitrary unit of one.

FIG. 4 illustrates: (A) a chart showing the expression of neuron-specific genes MAP2 and TUJ-1 is upregulated in BORG overexpressing cells (bars marked +BORG) after differentiation into cells with long processes; bars marked control contain RT-PCR-based expression analysis in vector transfected cells; (B) immunostaining assays indicating an upregulation in several neuronspecific proteins in BORG-overexpressing cells (panels marked +BORG) versus vectortransfected cells (Control); the area marked by the white rectangle is enlarged in the panel to the right; (C) a chart showing clustering of genes involved in various aspects of neuronal function and a global upregulation in BORG-overexpressing cells; the percentage of genes up or down-regulated in the microarray analysis is shown for each GO-miner cluster; (D) images showing overexpression of BORG in C3H10T1/2 cells results in differentiation into neuron-like cells; BORG overexpressing and vector-transfected cells (+BORG and Control, respectively) are shown after two weeks of incubation in low serum media; and (E) immunostaining for the neuron-specific marker MAP2 indicates the reprogramming of BORG-overexpressing C3H10T1/2 cells into neurons.

FIG. 5 illustrates: (A) a chart showing stable transfection of a plasmid containing the BORG gene (lane labeled BORG) versus the empty plasmid vector (lane labeled Vector) leads to an increase in the expression level of BORG as shown by RT-PCR; error bars reflect two standard deviations from 3 independent measurements; the expression level is shown in arbitrary units; and (B) images showing the overexpressed BORG transcripts are localized to nuclei, similar to the endogenously expressed BORG (FIG. 1B). A mock experiment without the addition of the FITC-labeled BORG probe was performed to determine the level of background signal.

FIG. 6 illustrates immunostaining assays for three glial markers, Olig2 (oligodendrocyte lineage transcription factor 2, oligodendrocyte marker), GFAP (glial fibrillary acidic protein, astrocytes marker) and CSPG (chondroitin sulfate proteoglycan 4, an oligodendrocyte progenitor marker). The immunostaining assays indicate no change in protein level between the wild type and BORG overexpression cells, even after prolonged exposures. The immunostaining pictures have an exposure time of 3 seconds.

FIG. 7 illustrates: (A) images showing morphological changes in BORG overexpression cells after 10 days of incubation in neuronal differentiation media; similarly treated control cells are shown; arrow points to a small myotube in the control cells; and (B) images showing the neuron-specific marker TUJ-1 is expressed in BORG overexpression cells after 4 days of incubation in neuronal differentiation media.

FIG. 8 illustrates: (A) immunoassays showing shRNA mediated knockdown of BORG in BORG overexpressing cells results in loss of expression of the neuronal marker MAP2; and (B) the shRNAmediated knockdown of BORG in BORG overexpressing cells results in loss of the ability to morphologically differentiate into neurons. +BORG: C2C12 cells that stably overexpress BORG from a transfected expression cassette. +BORG+shRNAs: C2C12 cells that stably overexpress BORG from a transfected expression cassette but were also stably transfected with a vector containing a shRNA construct against BORG.

### DETAILED DESCRIPTION

This application relates to compositions and methods of transdifferentiating non-neuronal mammalian cells into neuronal cells and methods for their use in the treatment of neurological disorders. It was found that BORG, a large non-protein coding RNA that is expressed in both embryonic and adult neurons, is required for the morphological and gene expression pattern alterations occurring during neuronal differentiation. When BORG RNA is expressed from a vector (e.g., plasmid vector) at levels more than twice the levels normally found in non-neuronal mammalian cells, such as myoblast and fibroblast cell lines, the cells undergo a profound morphological change involving development of long processes, development of a round cell body and formation of network-like interactions between processes of different cells. Biochemical analysis of gene expression in these cells indicated that unlike their parent cells, they express mature neuronal markers MAP2, TUJ-1, and the neural progenitor marker, NF200, Tau, synapsin, and nestin, markers for myelinated neurons. It was also found that the cells stop proliferation, another sign of neuronal transdifferentiation. Thus, overexpression of large non-coding BORG RNA can reprogram, or transdifferentiate, non-neuronal mammalian cells by interrupting a preset non-neuronal differentiation pathway and direct the cells to a neuronal progenitor state, from which they can differentiate into mature neurons.

An aspect of the application relates to method of transdifferentiating a non-neuronal mammalian cell into a neuronal cell. The method includes transfecting a non-neuronal mammalian cell with a nucleic acid that promotes the transdifferentiation of the non-neuronal mammalian cell into a neuronal cell. The nucleic acid can be substantially homologous to BORG RNA and in some instances promote over expression of BORG RNA in the non-neuronal mammalian cells.

The non-neuronal mammalian cells can be any mammalian cell capable of transdifferentiating into neuronal cells or cells capable of functioning as neuronal tissue following the overexpression of BORG RNA. The cells may originate from a subject into which they are implanted (reimplantation) or from elsewhere (transplantation).

The non-neuronal cells can include terminally differentiated or non-terminally differentiated cells, such as pluripotent adult or embryonic stem cells, multipotent cells, and totipotent cells. Examples of cells that can be used herein include but are not limited to fibroblasts, myoblasts, osteoblasts, chondroblasts, adipoblasts, B cells, T cells, dendritic cells, keratinocytes, adipose cells, epithelial cells, epidermal cells, chondrocytes, cumulus cells, glial cells, astrocytes, cardiac cells, esophageal cells, muscle cells, melanocytes, hematopoietic cells, macrophages, monocytes, and mononuclear cells.

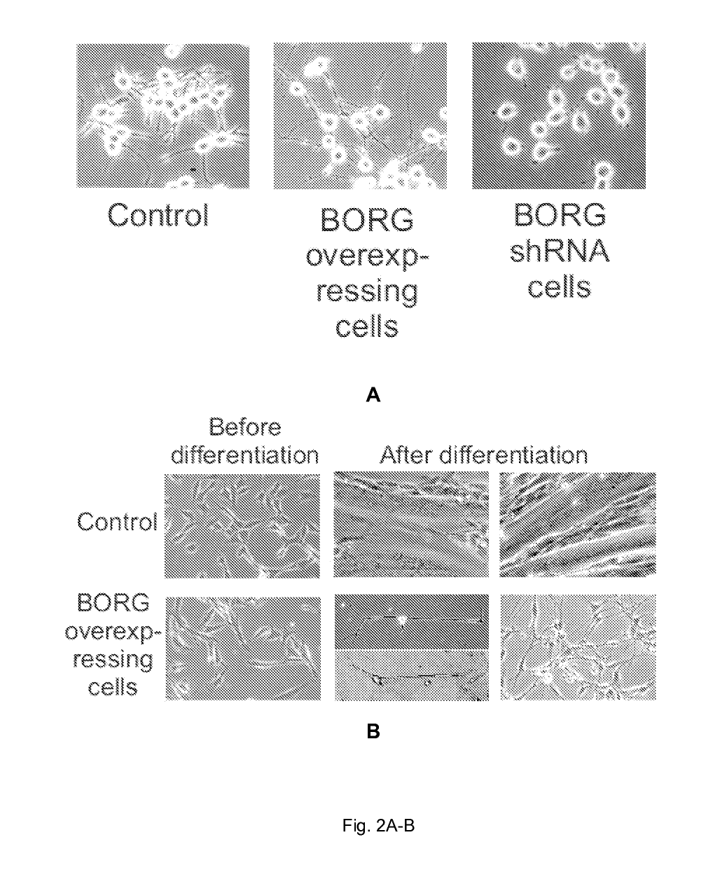

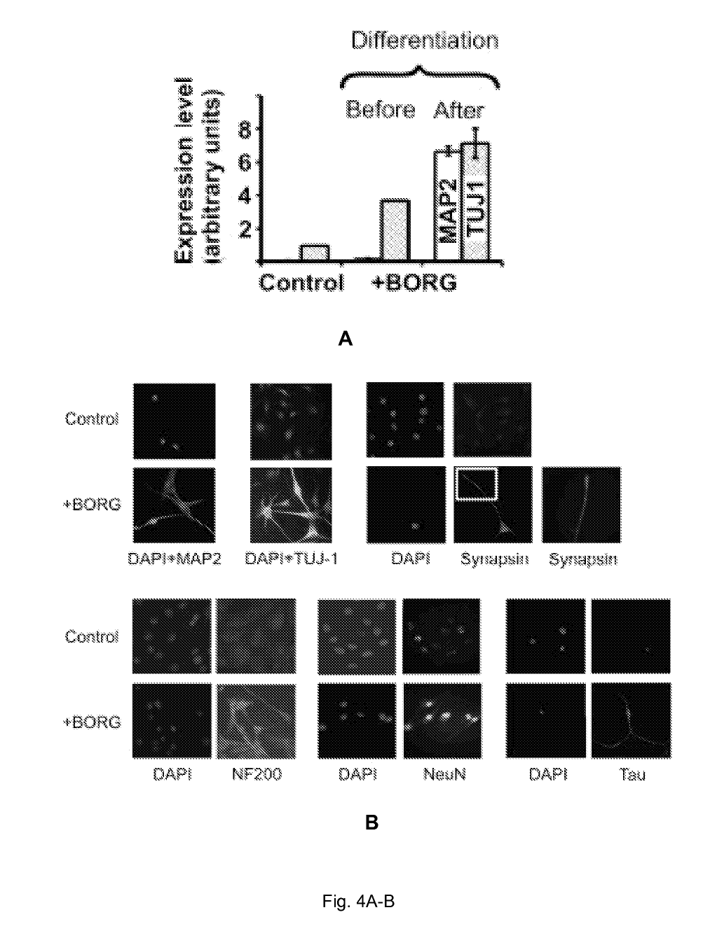

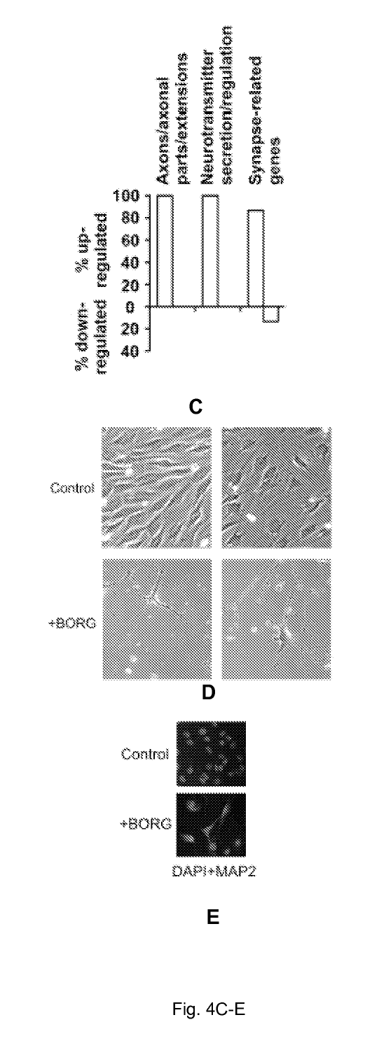

In some aspects, the method described herein can be utilized to transdifferentiate cells of mesodermic origin into a neuronal fate. Cells of mesodermic origin form skeletal muscle, the skeleton, the dermis of skin, connective tissue, the urogenital system, the heart, blood (lymph cells), and the spleen. As shown in the Example herein, the overexpression of BORG RNA can transdifferentiate at least two different mesodermic cell types into a neuronal fate. Specifically, it has been shown that both myoblasts and fibroblasts show neuron-like phenotypes once the level of BORG RNA is overexpressed in the cells (FIGS. 2 and 4). Therefore, the non-neuronal cells obtained for use in the present method can include cells of a mesodermic origin, such as but not limited to myoblasts, fibroblasts, osteoblasts, chondroblasts, or adipoblasts.

In one particular aspect, the non-neuronal cells are myoblasts. Myoblasts, as used herein, refers to primary cells derived from a muscle sample (either satellite cells surrounding the muscle fiber or the myogenic cells that arise from treating the muscle with Myoseverin), or commercially available cell lines, e.g., C2C12.

In another particular aspect, the non-neuronal cells are fibroblasts. Fibroblasts, as contemplated herein are cells found throughout the body that make the structural fibers and ground substance of connective tissue or commercially available cell lines, e.g., C3H10T1/2 embryonic fibroblasts.

Exemplary mammalian cells that can be transdifferentiated by the method of the application include but are not limited to human and non-human primate cells, ungulate cells, rodent cells, and lagomorph cells. Primate cells with which the method may be performed include but are not limited to cells of humans, chimpanzees, baboons, cynomolgus monkeys, and any other New or Old World monkeys. Ungulate cells with which the method may be performed include but are not limited to cells of bovines, porcines, ovines, caprines, equines, buffalo and bison. Rodent cells with which the method may be performed include but are not limited to mouse, rat, guinea pig, hamster and gerbil cells. Rabbit cells are an example of cells of a lagomorph species with which the method may be performed.

The non-neuronal mammalian cells can be obtained using various methods. In some aspects, the non-neuronal cells are obtained or isolated via any type of surgical procedure. The tissues containing the non-neuronal cells can be surgically removed from a subject via a biopsy. For example, both fibroblasts and myoblasts are highly abundant cells and are easily obtainable from patients by an outpatient biopsy. Alternatively, the non-neuronal cells can be obtained from an explant culture, where pieces of tissue are placed in growth media, and the cells that grow out are available for culture or from a stable cell line having the ability to proliferate indefinitely while maintained in culture.

Non-neuronal cells can be isolated for in vitro culture in several ways known in the art. For example, cells can be purified from blood or released from soft tissues by enzymatic digestion with enzymes such as collagenase, trypsin, or pronase. The cells may also be isolated using cell-sorting methods.

Non-neuronal cells obtained can be transdifferentiated without culturing or maintained in culture for use in the present methods. “Cultured” and “maintained in culture” are interchangeably used when referring to the in vitro cultivation of cells and include the meaning of expansion or maintenance of a cell population under conditions known to be optimal for cell growth.

Culture media for use in the method described herein is available as packed, premixed powders or pre-sterilized solutions. Commonly used media include MEM, DME, RPMI 1640, DMEM, Ham's F-10, Iscove's complete media or McCoy's Medium. Media culture may be further supplemented with 5-20% heat inactivated serum (e.g., 10% fetal bovine serum (FBS). In some aspects, the media can include a low serum cell culture medium wherein the serum is supplemented with no more than 3% serum. Other supplements to media typically include buffers, antibiotics, amino acids, sugars, and growth factors.

The non-neuronal mammalian cells obtained can be transfected by introducing a genetic construct including genetic material encoding functioning BORG RNA into the cells. “Genetic material” as used herein, refers to any material, which can encode for functioning BORG RNA, including but not limited to genomic DNA, cDNA and subfragments, splice variants, and variants thereof, including insertions, additions and deletions.

The genetic material can be substantially homologous to functional BORG RNA. By substantially homologous, it is meant the genetic material has at least about 50%, about 70%, about 80%, about 90%, about 95%, about 96%, about 97%, about 98%, about 99% or about 100% sequence identity with the nucleotide sequence of native BORG RNA.

In some aspects, the genetic material includes cDNA encoding BORG RNA. For example, SEQ ID NO: 1 corresponds to mouse cDNA encoding BORG RNA. In other aspects, the genetic material includes genomic DNA encoding BORG RNA. For example, SEQ ID NO: 2, corresponds to human genomic DNA encoding BORG RNA.

In other aspects, the cDNA can encode BORG RNA fragments that retain the transdifferentiation functionality of full length BORG RNA. BORG RNA fragments that can promote transdifferentiation of non-neuronal mammalian cells to neuronal cells or cells of neuronal lineage can include deletions or substitutions of nucleotides of BORG RNA and can be substantially homologous to BORG RNA. Functional BORG RNA fragments can be indentified using cell based assays described in the Example.

In one example, cDNA encoding a functional BORG RNA fragment can have the nucleotide sequence of SEQ ID NO: 7. SEQ ID NO: 7 includes nucleotides 1-535 and 735-2766 (536-734 deleted) of SEQ ID NO: 1. In another example, cDNA encoding a functional BORG RNA fragment can have the nucleotide sequence of SEQ ID NO: 8. SEQ ID NO: 8 includes nucleotides 1-734 and 1042-2766 (735-1041 deleted) of SEQ ID NO: 1. In a further example, cDNA encoding a functional BORG RNA fragment can have the nucleotide sequence of SEQ ID NO: 9. SEQ ID NO: 9 includes nucleotides 1-2129 and 2521-2766 (2130-2520 deleted) of SEQ ID NO: 1.

Additionally, it is well within the ability of the skilled artisan to synthesize BORG cDNA from genomic DNA for use in a nucleic acid construct described herein. This process typically includes synthesizing cDNA from mature (fully spliced) RNA using the enzyme reverse transcriptase.

The term “construct” as used herein, refers to a recombinant nucleic acid, generally recombinant DNA that has been generated for the purpose of the expression of a specific nucleotide sequence. Once the nucleic acid construct introduced into the non-neuronal cell, the BORG RNA that is encoded by the genetic material is produced by the cellular-transcription machinery of the cell resulting in the overexpression of the BORG RNA gene in the cell.

BORG RNA gene expression, as contemplated herein, is the process by which information encoded by the BORG RNA genetic material is used in the synthesis of a functional gene product. In non-protein coding genes, such as the BORG RNA gene, the gene product is not further translated and is considered a functional RNA. “Overexpression” as used herein, refers to artificial expression of a gene in increased quantity. Therefore, the term “overexpression of BORG RNA” as used herein, refers to the increased quantity of artificial BORG RNA in a cell.

It is to be understood that certain genetic material encoding BORG RNA contemplated herein, is unique to the mammalian species from which the cells are derived. Thus, the genetic material encoding BORG RNA provided to a cell can be relatively determined between mammalian species by the skilled artisan.

In certain embodiments, the BORG RNA provided to non-neuronal human cells can be genetic material (e.g., nucleic acids) substantially homologous to human BORG RNA. As used herein, the term “substantially homologous” means that the genetic material referred to is functionally the same as the genetic material endogenous to the identified mammalian species. Similarly, the term “genetic material substantially homologous to human BORG RNA” refers to genetic material that encodes for the BORG RNA that is functionally the same as BORG RNA encoded by an endogenous human BORG RNA gene.

Additionally, nucleic acid constructs for use in the method of the application may have expression signals such as a strong promoter, a strong termination codon, adjustment of the distance between the promoter and the cloned gene, and the insertion of a transcription termination sequence.

In certain aspects, the nucleic acid construct includes a nucleic acid substantially homologous to BORG RNA operably linked to a promoter to facilitate expression of the BORG RNA within a non-neuronal mammalian cell. The term “promoter” refers to a minimal sequence sufficient to direct transcription. “Promoter” is also meant to encompass those promoter elements sufficient for promoter dependent gene expression controllable for cell-type specific, tissue-specific or inducible by external signals or agents; such elements may be located in the 5′ or 3′ regions of the BORG RNA gene. The promoter may be a strong, viral promoter that functions in eukaryotic cells such as a promoter derived from cytomegalovirus (CMV), simian virus 40 (SV40), mouse mammary tumor virus (MMTV), Rous sarcoma virus (RSV), or adenovirus. In certain aspects, the promoter is a constitutive CMV promoter.

Alternatively, the promoter used may be tissue-specific, cell type-specific promoter, or a strong general eukaryotic promoter, such as the actin gene promoter. In another aspect, the promoter is a regulated promoter, such as a tetracycline-regulated promoter, expression from which can be regulated by exposure to an exogenous substance (e.g., tetracycline).

The nucleic construct may also include sequences in addition to promoters, which enhance expression in the target cells. For example, a nucleic acid substantially homologous to BORG RNA can be operably linked to a polyadenylation signal sequence. The polyadenylation signal sequence may be selected from any of a variety of polyadenylation signal sequences known in the art. An exemplary polyadenylation signal sequence is the SV40 early polyadenylation signal sequence. In addition, the nucleic acid construct may also include one or more introns, where appropriate, which can increase levels of expression of the BORG RNA, particularly where the nucleic acid encoding BORG RNA is a cDNA (e.g., contains no introns of the naturally-occurring sequence).

In some aspects, the nucleic acid construct may include a reporter gene to aid in identification of cells containing and/or expressing the nucleic acid construct provided to the cells. The reporter gene preferably can include a light emitting reporter gene, for example one that encodes a protein that is fluorescent. Accordingly, a reporter gene for use herein can be a green fluorescent protein (GFP) and light emitting derivatives thereof. GFP is from the jellyfish Aquorea victoria and is able to absorb blue light and re-emits an easily detectable green light. GFP may be advantageously used as a reporter because its measurement is simple and reagent free and the protein is non-toxic.

In other aspects, the nucleic acid construct may include a marker to aid in the selection of non-neuronal cells containing the nucleic acid construct. Alternatively, the marker may be co-transfected with the nucleic acid construct. Typically, selectable markers provide for resistance to antibiotics such as but not limited to tetracycline, ampicillin, hygromycin, and neomycin or thymidine kinase.

Introduction of one or more of the nucleic acid construct(s) encoding BORG RNA can be achieved using a variety of gene transfer protocols permitting transfection of heterologous nucleic acid into the cells. The term “transfection” refers to a permanent or transient genetic change, preferably a permanent genetic change, induced in a cell following incorporation of foreign nucleic acid (e.g., DNA or RNA exogenous to the cell). Genetic change can be accomplished either by incorporation of the new nucleic acid into the genome of the host cell, or by transient or stable maintenance of the new DNA as an episomal element. A cell has been “transfected” when the nucleic acid construct has been introduced inside the cell membrane using any technology used to introduce nucleic acid molecules into cells.

In an exemplary embodiment, the non-neuronal mammalian cells are transfected with one or more nucleic acid constructs, wherein the nucleic acid construct comprises a constitutive promoter operatively linked to a nucleic acid substantially homologous to BORG RNA.

A number of transfection techniques are well known in the art and are disclosed herein. See, for example, Graham et al., Virology, 52: 456 (1973); Sambrook et al., Molecular Cloning, a laboratory Manual, Cold Spring Harbor Laboratories (New York, 1989); Davis et al., Basic Methods in Molecular Biology, Elsevier, 1986; and Chu et al., Gene, 13: 197 (1981). Such techniques can be used to introduce one or more nucleic acid constructs described herein into the non-neuronal cells.

In some aspects, the nucleic acid construct can be introduced in vitro into the non-neuronal cell using a vector. The term “vector” as used herein, refers to any compound, biological or chemical, which facilitates transformation of a target cell with a DNA of interest. Exemplary vectors include but are not limited to viral vectors, plasmids, cosmids, and yeast artificial chromosomes. The precise vector and vector formulation used will depend upon several factors, such as the size of the nucleic acid construct to be transferred and the delivery protocol to be used. The nucleic acid construct can also be introduced as infectious particles, e.g., DNA-ligand conjugates, calcium phosphate precipitates, and liposomes.

In general, viral vectors used in accordance with the method described herein are composed of a viral particle derived from a naturally occurring virus, which has been genetically altered to render the virus replication-defective and to deliver a recombinant gene of interest for expression in a target cell. Numerous viral vectors are well known in the art, including, for example, retrovirus, adenovirus, adeno-associated virus, herpes simplex virus (HSV), cytomegalovirus (CMV), vaccinia and poliovirus vectors. The viral vector may be selected according to its preferential infection of the non-neuronal cells targeted.

Where a replication-deficient virus is used as the viral vector, the production of infectious virus particles containing either DNA or RNA corresponding to the nucleic acid construct can be achieved by introducing the viral construct into a recombinant cell line, which provides the missing components essential for viral replication. Transformation of the recombinant cell line with the recombinant viral vector will not result in production or substantial production of replication-competent viruses, e.g., by homologous recombination of the viral sequences of the recombinant cell line into the introduced viral vector. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus).

The nucleic acid construct may be introduced into a cell using a non-viral vector. “Non-viral vector” as used herein is meant to include naked DNA (e.g., DNA not contained within a viral particle, and free of a carrier molecules such as lipids), chemical formulations comprising naked nucleic acid (e.g., a formulation of DNA (and/or RNA) and cationic compounds (e.g., dextran sulfate, cationic lipids)), and naked nucleic acid mixed with an adjuvant such as a viral particle (e.g., the DNA of interest is not contained within the viral particle, but the formulation is composed of both naked DNA and viral particles (e.g., adenovirus particles) (see, e.g., Curiel et al. 1992 Am. J. Respir. Cell Mol. Biol. 6:247-52). Thus, “non-viral vector” can include vectors composed of nucleic acid plus viral particles where the viral particles do not contain the nucleic acid construct within the viral genome.

In some aspects, a liposome non-viral vector can be used to introduce the nucleic acid construct to the non-neuronal cell. Suitable liposomes for use in the method described herein comprise a mixture of lipids, which bind to the nucleic acid construct and facilitate delivery of the construct into the cell. Liposomes that can be used include include DOPE (dioleyl phosphatidyl ethanol amine), CUDMEDA (N-(5-cholestrum-3-beta.-ol 3-urethanyl)-N1,N1-dimethylethylene diamine).

Once the nucleic acid construct is introduced into a non-neuronal cell (e.g., by transfection), the cell can be cultured in a suitable growth medium to allow for transdifferentiation or administered directly to a subject, which is being treated with the transdifferentiated cell. After the cells are transfected with the BORG RNA, the induction of differentiation of the transfected non-neuronal cells into neuronal cells can occur quickly. As discussed in the Example below, when the expression level of BORG RNA is raised in a non-neuronal cell, transdifferentiation into a neuronal cell can occur in a matter of days.

Overexpression of BORG RNA in a population of non-neuronal cells results in the formation of a population of newly transdifferentiated neuronal cells. The population of transdifferentiated neuronal cells can have morphological, physiological and/or immunological features of a population of neuronal cells. These features may include expression of one or more specific marker(s). In some aspects, the population includes a plurality of transdifferentiated neuronal cells expressing at least one neural-specific antigen selected from the group consisting of MAP2, TUJ-1, NF200, Tau, synapsin, and nestin.

The population of transdifferentiated neuronal cells can be maintained in culture. The cell culture can be maintained under culture conditions including suitable temperature, pH, nutrients, and proper growth factors known in the art that favor the in vitro propagation of neuronal cells.

In an aspect of the application, the transdifferentiated cell(s) can be manipulated under culture conditions in vitro in the presence of specific exogenously supplied signal molecules, or in vivo within specific microenvironments, into diverse neuronal cell types as defined by the practitioner's operative criteria. For example, the cell culture can be further manipulated to express additional or different neural-specific specific-markers in the presence of specific exogenously supplied signal molecules. In addition, it is also contemplated herein that different non-neuronal cell types may be transdifferentiated into specific neuronal cell classes (e.g., sensory neuronal cells, motoneuronal cells, or interneuronal cells) under suitable culture conditions or in specific microenvironments.

In some aspects, it may be advantageous to verify that a non-neuronal cell has transdifferentiated into a neuronal cell. Transdifferentiation can be detected by any means known in the art including, e.g., detecting expression of neuronal cell type-specific marker proteins, observing morphological changes of cells and detecting fluorescent intensity of cells after treatment with a fluorescent styryl neuron dye.

In some aspects, non-neuronal cells transfected with the nucleic acid construct described herein may be assayed to detect a neuronal cell surface marker using well known methods. For example, neuronal cell markers indicating transdifferentiation include at least one neural-specific antigen selected from the group consisting of MAP2, TUJ-1, NF200, Tau, synapsin, and nestin (FIG. 4). The level of particular cell-specific markers can be conveniently measured using immunoassays, such as immunocytochemical analysis, western blotting analysis, ELISA and so on with an antibody that selectively binds to the particular neuronal cell markers.

Morphological changes of cells indicative of transdifferentiation become more evident steadily over time. Morphological changes can include elongated, sometime branched process formation, usually at opposite poles of the cell (FIG. 2A). The processes from a number of different transdifferentiated cells can form interconnected network, with processes from one cell frequently ending on another cell's body or processes (FIG. 2B). Morphological changes may be detected using any methods known to those of skill in the art. Typically, morphological changes of the cells are visually detected using a light microscope.

Transdifferentiation may be also detected by measuring fluorescent intensity of cells after incubation with FM 1-43 in the presence of 100 mM KCl. At a high concentration of K+, FM1-43 enters the neuron cells when the synaptic vesicles are recycled back into the neuron after depolarization. Thus, transdifferentiated neuron cells exhibit a high fluorescence signal.

Additional physiological features may be used to verify transdifferentiation. These features can include: synthesis of neurotransmitter(s) (e.g., dopamine or GABA); the presence of receptors for neurotransmitter(s); membrane excitability and/or developmental response to particular cytokines or growth factors; and lack of mitotic activity under cell culture conditions can be indicative of neuronal transdifferentiation.

As described above, it has been shown that the inventive transdifferentiated neuronal cells and cell cultures containing populations of transdifferentiated neuronal cells display morphological and physiological features of neurons. Thus, it is further contemplated that the newly created transdifferentiated neurons can be provided in therapeutic compositions and used to replace lost and defective cells in cell therapies aimed at alleviating neurological disorders and diseases.

Another aspect of the application relates to a method of treating a neurological disorder in a subject by administering a therapeutically effective amount of the transdifferentiated neuronal cells to the subject. The therapeutically effective amount of transdifferentiated neuronal cells to be administered to a subject can be determined by a practitioner based upon such factors as the levels of transdifferentiation achieved in vitro, the mode of administration, the particular neurological disorder, and/or the number of cells that survive implantation.

As described above the non-neuronal cells may originate from a subject into which they are implanted (reimplantation) or from elsewhere (transplantation). In some aspects, a subject is administered transdifferentiated neuronal cells derived from the subject's own body because the risk of transmission of an infection such as HIV is eliminated and the risk of triggering an immune system-mediated rejection reaction is reduced.

An advantage of the transdifferentiated neuronal cells and neuronal cell cultures is that there is no need for cell expansion, as is required with stem cell technology used to generate neurons for cell therapies. Thus, in one aspect, the transdifferentiated neuronal cells can be directly administered to a subject without requiring a step for cell expansion.

In general, the transdifferentiated neuronal cells are administered (e.g., implanted) into the mammalian subject by methods well known in the art. The engraftment of the transdifferentiated neuronal cells may be monitored by examining the subject for classic signs of graft rejection, i.e., inflammation and/or exfoliation at the site of implantation, and fever, and by monitoring neuronal function.

The transdifferentiated cells may be introduced into the subject by any suitable route whether that route is enteral or parenteral, for example, intravenous or intramuscular. The transdifferentiated neurons are preferably administered in an area of a neurological disorder, and in a manner that minimizes surgical intervention in the subject (e.g., stereotactic injection). In one exemplary embodiment, the transdifferentiated neuronal cells may be administered directly into the brain, preferably a brain lesion site.

The term “neurological disorder” refers to diseases and disorder of the peripheral nervous system, such as peripheral nerve injuries, peripheral neuropathy and localized neuropathies, and central nervous system diseases. A central nervous system disease as contemplated herein may include but is not limited to Alzheimer's disease, Parkinson's disease, cerebral palsy, Huntington's disease, amyotrophic lateral sclerosis, elderly dementia, Tay-Sach's disease, Sandhoffs disease, Shy-Drager syndrome, Hurler's syndrome, epilepsy, tumor, multiple sclerosis and Krabbe's disease. Further, conditions which may be treated can include mechanical and neurotraumatic disorders, such as spinal cord disorders, spinal cord injury, head trauma stroke, cerebrovascular diseases, and birth-induced traumatic central nervous system injury.

In some aspects, after transdifferentiating the non-neuronal cells, the newly created transdifferentiated neuronal cells are allowed to form functional connections either before or after a step involving administration of the transdifferentiated neurons to the subject. This aspect may be particularly advantageous in the treatment of Parkinson's disease. In many neurological disorders, unlike Parkinson's disease, the underlying cause of symptoms cannot be attributed to a single factor. This condition may render the therapeutic approach of introducing a single neuronal cell type replacement less effective. Rather, replacement of the lost or diseased host neuronal cells, or even lost or diseased neuronal networks in a subject by a healthy functioning neuronal networks is required.

As described above, the method of the application enables the practitioner to develop different types of neuronal cells (e.g., motor neurons, interneurons, and sensory neurons). These newly formed neurons can be cultured separately, or together, to stimulate formation of functional neuronal networks that can be used for replacement therapies. Alternatively, different types of neurons can be transplanted and induced to form functional connections between themselves and host neurons, in situ, in the brain or in the spinal cord.

In one example, the method of the application can be used to treat Parkinson's disease in a subject. In the method, non-neuronal cells can be obtained from the affected subject (e.g., myoblasts or fibroblasts). The cells can then be transfected to overexpress BORG RNA and allowed to transdifferentiate into neuronal cells. The resulting transdifferentiated neuronal cells can then be implanted into the patient's striatum or brain. The cells are typically implanted bilaterally in the caudate nucleus and putamen by using Magnetic Resonance Imaging (MRI)-guided stereotactic techniques. After the implantation surgery, the implanted cells may secrete dopamine in situ alleviating the subject's Parkinson's disease symptoms.

In another example, the method of the application can be used to treat Alzheimer's disease in a subject. In the method, non-neuronal cells are first obtained from the affected subject. The cells can then be transfected with a nucleic acid construct to overexpress BORG RNA and allowed to transdifferentiate into neuronal cells. The cells can then be implanted in a lesion site in the subject's brain, or the transdifferentiated cells are cultured so as to facilitate development of the cells into neuronal tissue prior to implantation. Alternatively, cells from another subject (the “donor”) can be transfected to overexpress BORG RNA, and the cells subsequently implanted in the affected subject to provide neuronal tissue, or the transdifferentiated cells cultured so as to facilitate development of the cells into neuronal tissue prior to implantation.

The compositions and methods of the application will now be described in greater detail in the following non limiting Example.

### EXAMPLE

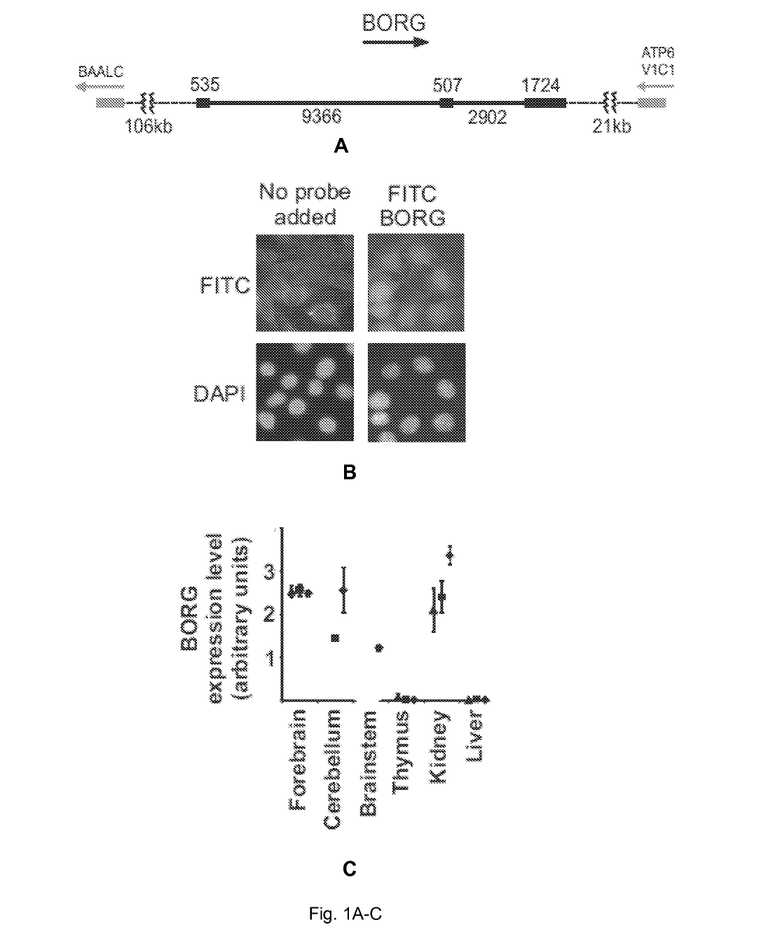

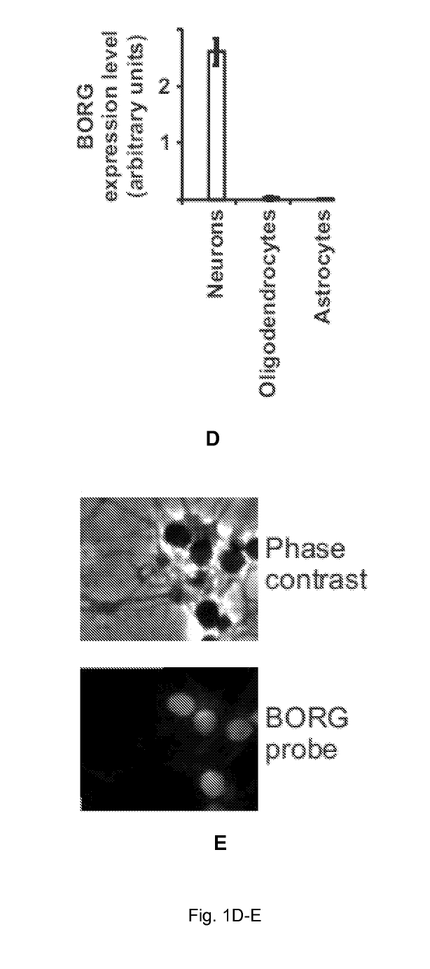

To gain insight into the impact and scope of function of long non-coding RNAs in higher eukaryotes, we analyzed the cellular role of a long mRNA-like noncoding RNA, BORG, which was originally described as a BMP2-responsive gene in the mouse myoblast C2C12 cell line. BORG is a 2766 nucleotide long mRNA-like transcript and is both spliced and polyadenylated (FIG. 1A). Extensive expression analyses indicated that BORG does not have any protein coding potential. Bioinformatics analysis of the mouse BORG transcript confirmed this conclusion by demonstrating that the small predicted ORFs were not conserved across even very short evolutionary distances and further showed a high rate of non-synonymous to synonymous mutations compared to known protein-coding ORFs. The BORG gene is found in all sequenced mammalian genomes and shows moderate sequence conservation, as is the case with other long non-coding RNAs.

Analysis of the genomic locus of BORG on mouse chromosome 15qB3.1 indicated that BORG did not overlap in the sense or antisense direction with any known transcripts and thus, was not likely to act via antisense mechanisms in cis (FIG. 1A). The closest known genes to BORG were 21 and 106 Kilobases away in antisense converging and diverging orientations, respectively (FIG. 1A), making transcriptional interference between BORG and the two transcripts unlikely. Our analysis also indicated that BORG was not a miRNA precursor. Thus, BORG performs any potential cellular function it may have as a long non-coding RNA through an unknown mechanism.

To begin to determine the cellular function of BORG, we first confirmed that it is indeed induced in response to BMP2 in C2C12 cells. In the absence of BMP2, BORG is expressed at a low basal level and the analysis of subcellular localization of BORG by in situ hybridization before and after induction by BMP2 indicated that BORG is mainly localized in the nuclei (FIG. 1B). While BORG expression was detectable in C2C12 myoblasts, its expression level was much lower or not detectable in a number of other cell lines and the shRNA-mediated knock down of the expression of BORG did not interfere with cellular viability, suggesting that BORG is not essential for viability. To determine the cell types in which BORG might perform a function, we analyzed the tissue expression pattern of BORG during developmental stages in the mouse. Interestingly, BORG showed elevated expression levels in all neural tissues tested throughout fetal development and in adults, including forebrain, cerebellum, brainstem and spinal cord (FIG. 1C). BORG expression was not detectable in other tested tissues with the exception of kidneys (FIG. 1C), suggesting that its expression in neural tissues may have a functional significance. To determine whether the observed expression of BORG in neural tissues is restricted to a certain cell type, we isolated RNA from cultured primary neurons, astrocytes and oligodendrocytes, the major cell types found in forebrain, and analyzed them for BORG expression. Significantly, primary neurons showed robust BORG expression, while neither primary astrocytes nor oligodendrocytes detectably expressed BORG (FIG. 1D). In situ hybridization assays on primary neurons further confirmed the expression of BORG in these cells (FIG. 1E). Taken together, these results raised the possibility that BORG might play a role in differentiation, function or maintenance of neuronal cells.

As a first step toward determining the cellular function of BORG, we asked whether the expression of BORG had any effect on neuronal differentiation or function. To this end, we used Neuro2A (N2A) cells, a neuroblastoma cell line widely used as a model for neuronal differentiation. We made stably transfected N2A cell lines that contained either an additional copy of the spliced transcript of BORG or shRNA constructs against BORG under the control of a constitutive CMV promoter. In the wild type N2A cells or cells transfected with the plasmid vector or scrambled shRNA constructs, induction of neuronal differentiation resulted in a marked increase in length of the short neurites of pre-differentiation N2A cells, which is indicative of their differentiation into neurons (FIG. 2A). Interestingly, BORG knock down cells did not show an increase in the number or length of neurites after the induction of neuronal differentiation, indicating that shRNA-mediated knockdown of BORG in N2A cells inhibited their ability to differentiate (FIG. 2A). The shRNA cells on average had 1-1.3 neurites per cell and less than 5% of them were more than twice the cell diameter in length, an indicator of differentiation in N2A cells, while control cells had 2.5 neurites per cell and ˜50% of cells had one or more neurites longer than twice the diameter of the cell. On the other hand, increased expression level of BORG enhanced the elongation of the N2A neuritis compared to wild type cells (FIG. 2A). BORG overexpressing cells had 3 neurites per cell on average, with close to 90% of cells having at least one neurite that was longer than twice the cell diameter. Together, these results suggest that BORG RNA is required for the conversion of the pre-differentiation N2A cells to fully-differentiated neurons. Further, the above data also indicated that at least part of the cellular function of BORG was mediated by the transcript itself, rather than the mere act of transcription from its locus or induction of chromatin remodeling or transcriptional interference in cis which has been observed in the case of several other long ncRNAs.

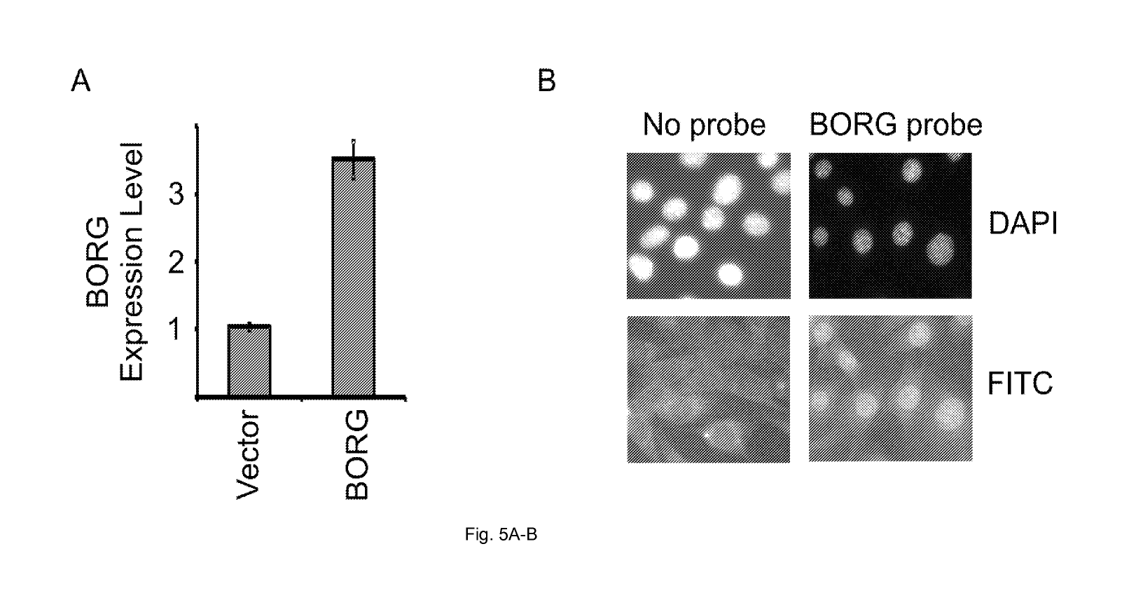

Since the above experiments indicated a positive correlation between BORG expression levels and induction of differentiation in neuronal cells, we set out to determine whether overexpression of BORG had any effect on differentiation in nonneuronal cells. To this end, we made stable C2C12 cell lines that contained a copy of the spliced transcript of BORG and showed a 3-4 fold increase in the cellular level of BORG transcript (FIG. 5). To ensure that any observed results were due to overexpression of BORG and not disruption of another gene at the integration site, we analyzed the gene expression pattern and morphology of over 30 distinct colonies that were each derived from a single stably transfected cell and thus, had different integration sites. C2C12 cells are of myoblast origin and differentiate into myotubes, but they can also be induced to differentiate into a number of other mesodermal lineage fates, including chondrocytes, adipocytes, and osteoblasts.

The initial analysis of BORG overexpressing cells showed an altered growth pattern, with an arrest in proliferation once a certain cell density was reached. To determine if overexpression of BORG affects the differentiation ability of the C2C12 cells, we incubated the BORG overexpressing stable cell lines and control C2C12 cells in muscle differentiation media. As expected, control C2C12 cells and vector-transfected control stable cell lines efficiently formed multinucleated myotubes (FIG. 2B). Strikingly, all BORG overexpression cell lines showed a dramatically different morphology (FIG. 2B). Rather than fusing together and forming myotubes, they remained separated as individual cells and formed round cell bodies with elongated, sometimes branched processes, usually at opposite poles of the cell (FIG. 2B). With longer incubations, the processes from different cells formed a highly interconnected network, with processes from one cell frequently ending on another cell's body or processes (FIG. 2B).

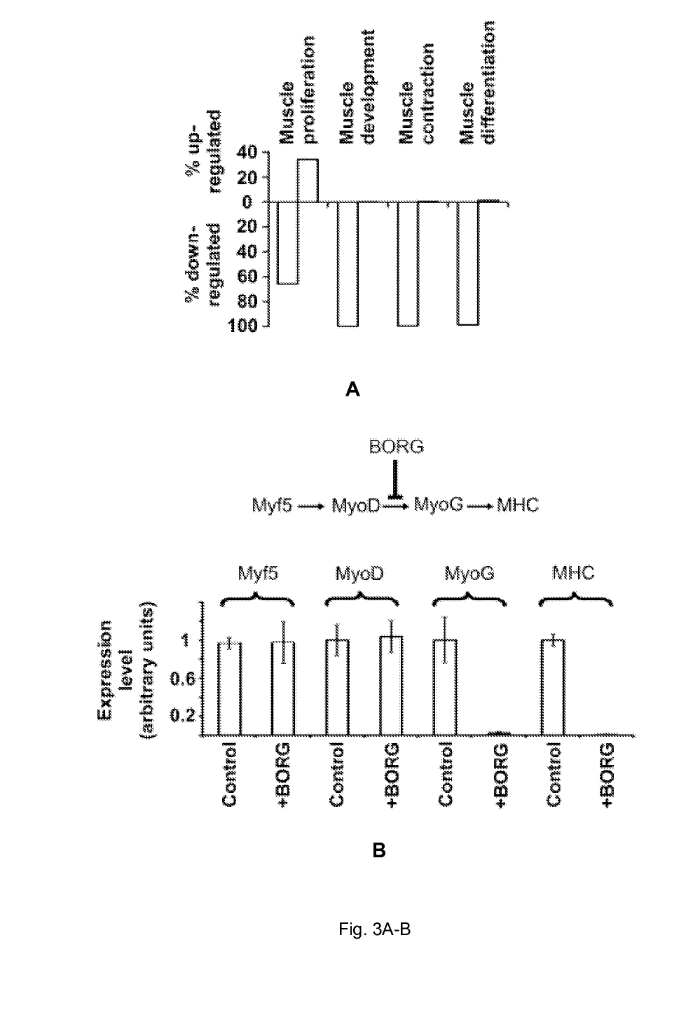

Analysis of the global gene expression profile of BORG overexpressing cells compared to vector-transfected cells using microarrays showed a significant downregulation of a large number of genes involved in muscle contractile function, the myogenic proliferation, differentiation and development pathways, consistent with a global change in the cellular differentiation program (FIG. 3A). The expression of several muscle-specific genes involved in terminal myogenic differentiation and muscle function was down-regulated ten to fifty fold, including the key commitment factor in myogenic differentiation, MyoG. Further analysis of the myogenic differentiation pathway by RT-PCR indicated that as suggested by the global expression analyses, the level of transcription factors Pax7, MyoD and Myf5, the upstream regulators of myogenic differentiation, was not changed as a result of BORG overexpression (FIG. 3B). However, the expression of MyoG, which is induced by MyoD and commits the cells to myogenic differentiation, and the downstream factors such as myosin heavy chain were completely absent in BORG overexpression cells (FIG. 3B). Taken together, these results indicated that the overexpression of BORG in C2C12 myoblasts results in interruption of the myogenic differentiation pathway at the step of induction of MyoG by its upstream transcription factor, MyoD.

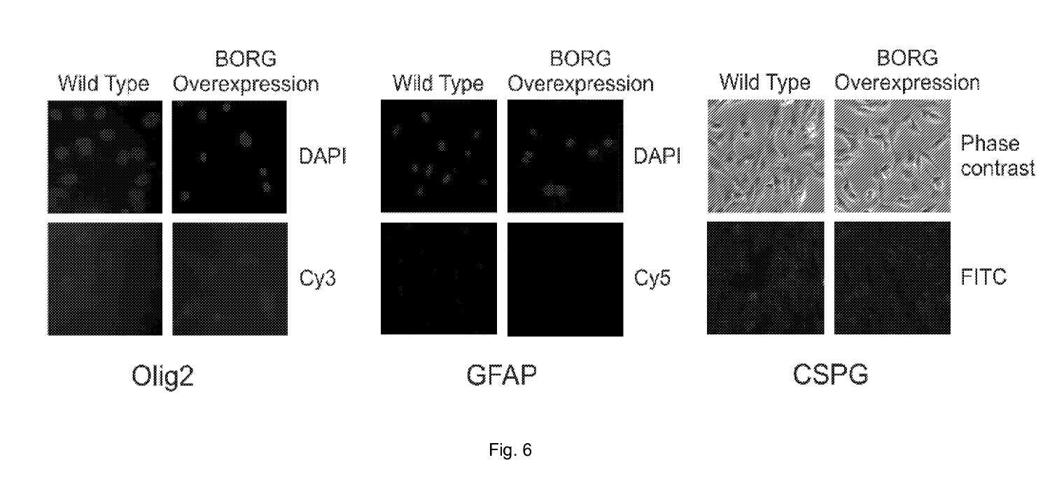

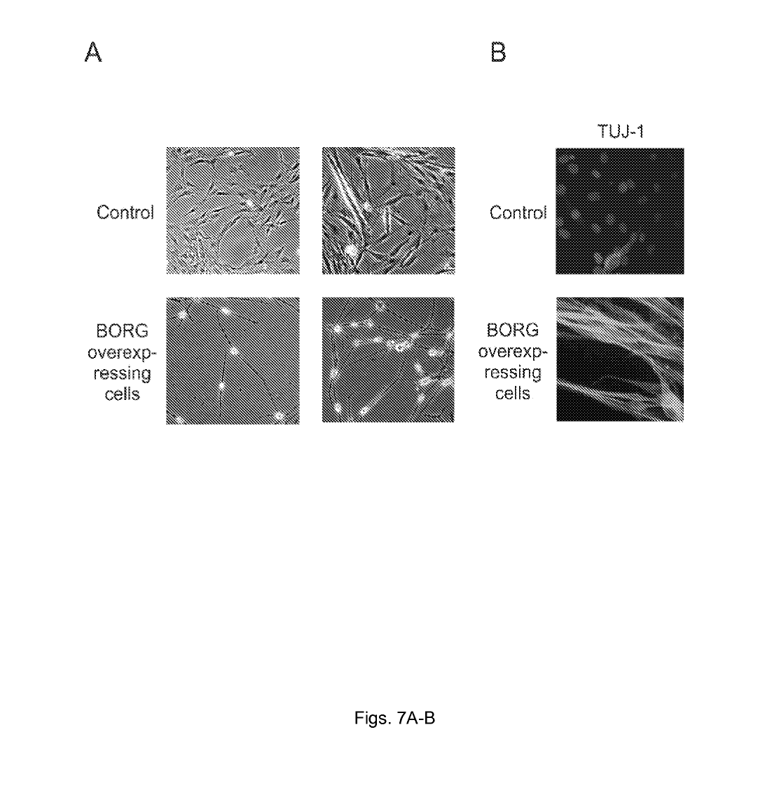

Concomitant with the down-regulation of genes involved in muscle differentiation and function, BORG overexpressing cells showed an increase in the expression of genes found in neuronal cells (FIG. 4A). RT-PCR assays on several neuronal differentiation markers, including MAP2 and TUJ-1, confirmed an increase in their expression in BORG overexpression cells, especially after their differentiation into cells with elongated, branched processes (FIG. 4A). Further, immunostaining against several neuronal markers on the BORG overexpression cells before and after differentiation showed a parallel increase in the protein expression level of mature neuronal markers MAP2, TUJ-1, NF200, tau, NeuN and synapsin (FIG. 4B), which was not observed in control or vector-transfected cells. MAP2, TUJ-1, tau, NeuN and synapsin are highly specific to neuronal tissues and their expression is considered indicative of neuronal differentiation. Consistent with a BORG-induced neuronal reprogramming, the level of expression of genes involved in the development and function of axons and axonal guidance, synapses and neurotransmitters showed a marked upregulation, while the expression level of a number of known inhibitors of axonal growth was strikingly reduced (FIG. 4C). In contrast to the neuronal markers, expression of glial markers such as GFAP, CSPG, A2B5 and Olig2 was not detectable in BORG overexpression cells or control cells (FIG. 6). Incubation of BORG overexpressing cells and control cells in previously characterized neuronal differentiation media also resulted in similar morphological and gene expression pattern changes in BORG overexpressing cells, while control cells did not show any observable changes (FIG. 7).

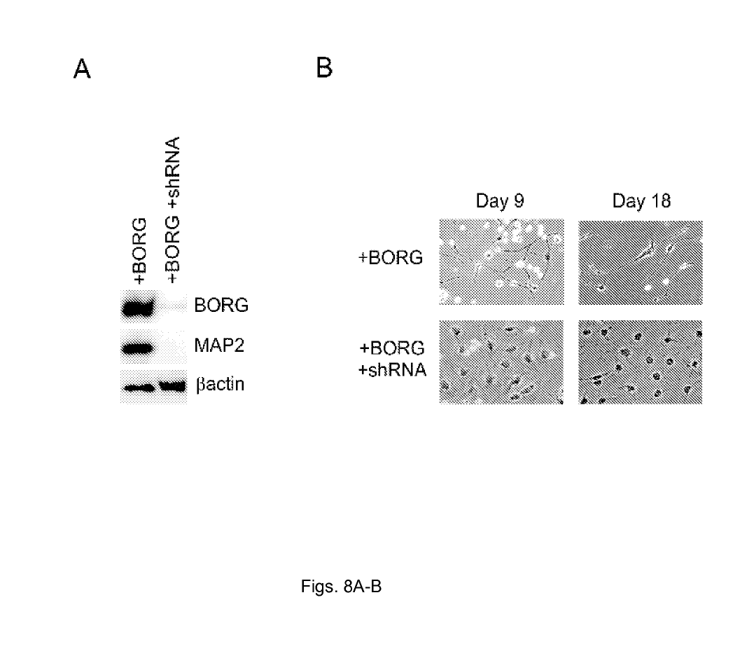

Together, these data indicate that overexpression of BORG, a non-coding RNA that is physiologically expressed in neuronal tissues, resulted in reprogramming of the gene expression pattern and induction of morphological changes characteristic of neuronal cells in C2C12 myoblasts. To further ensure that the observed morphological and gene expression pattern changes were the result of BORG overexpression, we stably transfected BORG overexpressing cells with shRNA constructs against BORG and monitored their morphology and gene expression pattern. Along with the shRNAmediated reduction in the level of BORG, these double-transfected cells lost their ability to morphologically differentiate into neurons and the level of expression of neuronal markers MAP2 and TUJ-1 was reduced to background (FIG. 8).

To determine if the observed reprogramming was specific to C2C12 myoblasts, we derived stable cell lines that overexpressed BORG in C3H10T1/2 embryonic fibroblasts. C3H10T1/2 cells can be induced to differentiate into lipocytes and several other mesodermal cell types. Intriguingly, upon incubation in low serum media, the BORG overexpressing C3H10T1/2 cells, and not the control or vector transfected cells, showed morphological changes similar to those observed with C2C12 cells overexpressing BORG (FIG. 4D). Analysis of the gene expression pattern of these cells indicated the upregulation of the neuronal differentiation marker MAP2, similar to what had been observed with the C2C12 cells (FIG. 4E). Thus, BORG RNA can reprogram at least two different mesodermal cell types into a neuronal fate. Taken together with the cell typespecific expression of BORG in primary neurons and neural tissues, these data indicate that the BORG RNA is a non-coding RNA regulator of neuronal differentiation pathways.

Global analyses of the mammalian transcriptomes suggest that the vast majority of the mammalian genomes are transcribed into non-protein-coding RNAs. Our data demonstrates that a member of this class of transcripts plays a dominant role in cellular reprogramming and cell fate specification by diverting cells of mesodermal origin into a neuronal fate, underscoring the power of long non-coding RNAs in regulating cellular fate and differentiation. The tissue- and cell type specific expression of BORG in neurons and the requirement of BORG for neuronal differentiation in model systems suggest that BORG plays a critical role in physiological differentiation of neural tissues in mammalians. The high efficiency of reprogramming via BORG overexpression indicates that the use of regulatory non-coding RNAs may provide an alternative technology to the existing induced pluripotent stem cell (iPS)-based techniques in regenerative medicine.

### Materials and Methods

### Cell Culture

### C2C12 Cells

The C2C12 murine myoblast cell lines used were kind gifts from Tim Nilson (originating from ATCC) and Nikki Harter (early passage C2C12 stock), both from Case Western Reserve University. The cells were maintained in Dulbecco's modified essential media (DMEM) (Invitrogen) supplemented with 15% fetal bovine serum (Invitrogen), penicillin (100 U/ml) and streptomycin (100 μg/ml) at 37° C. in a humidified atmosphere containing 5% CO₂. Cells were passaged at ˜55% confluency level. For all experiments, cells were seeded at a density of 1.0×10⁴ cells/cm² and the experiments were initiated after cells reached the confluency of 75%. In order to induce differentiation, once the cells were grown to 75% confluence the media was changed to the myogenic differentiation medium, consisting of DMEM plus 2% heat inactivated horse serum (Invitrogen) followed by incubation for 5-10 days. When indicated, a designated neuronal differentiation medium was used instead of the above media which contained 1% glutamax (100×), 1% N2 supplement (100×), 2% B27 Supplement (50×), cAMP 0.5 μM, 10 ng/ml of GDNF and BDNF.

### F9 Cells

F9 embryonic carcinoma cells (Kind gift of Philip Howe) were grown in DMEM supplemented with heat inactivated 10% calf serum and 2 mM glutamine at 37° C. Cells were harvested 72 h after plating and the total RNA was isolated by TRIzol, as described below.

### N2A Cells

N2a murine neuroblastoma Cells were grown in Dulbecco's Modified Eagle's Medium (DMEM, Invitrogen) supplemented with 10% fetal bovine serum (FBS, Invitrogen), 1 mM glutamine, penicillin G (100 U/ml) and streptomycin (100 μg/ml) at 37° C. in a humidified atmosphere with 5% CO₂ (1-5). Once the cells reached 80-90% confluence, they were transfected with 2 μg/mL of a vector containing the sequence of the spliced transcript of BORG or a shRNA against BORG (see below) and G418 resistance gene using Lipofectamine 2000 (Invitrogen) according to the manufacturer's instructions. Cells transfected with a vector that did not contain any inserts were used as control. After 3 days of incubation, the cells were switched to a medium supplemented with 500 μg/ml G418. The medium was changed every 3 days during the selection of viable cells. After 2 weeks, isolated colonies of viable cells were selected using cloning disks. The isolated clones were cultured in a medium supplemented with 500 μg/ml G418 for another 2 weeks and the viable cells were re-plated at low density, followed by a second round of colony selection to ensure the homogeneity of the selected clones. At least 8 stably transfected clones were obtained for each transfected plasmid and characterized. Two clones were typically chosen for more in-depth characterization.

To study neuronal differentiation, cells were seeded in 6-well culture plates at a density of 10,000 cells/well. After 24 hours, the cells were induced to differentiate by the replacement of the growth medium with DMEM containing 20 μM retinoic acid plus 2% FBS. The medium was refreshed every 24 hours. The morphology of cells was examined under a Zeiss light microscope on a daily basis. Cells having one or more neurites of a length more than twice the diameter of the cell body were defined as differentiated using the IimageJ software. To determine the percentage of differentiated cells, two controls (wild type cells and vectortransfected cells), two different clones of shRNA-mediated BORG knock down cells and two BORG overexpressing clones were visually inspected for the number of neurites and their length, Only cells that were well-separated from other cells and thus, their number of neurites could be determined with certainty were counted, although these cells seemed representative of the entire population on visual inspection. The total number of neurites and the number of neurites that were at least twice as long as the cell diameter were determined for 100 cells from each group. The difference between the two controls or between the two shRNA or BORG overexpressing cell lines was less than 5%.

### C3H10T1/2 Cells

C3H10T1/2 cells were generously provided by Drs. Keith Yamamoto and Ed Stavenezer. The cells were grown to confluence in high-glucose DMEM supplemented with 1 mM pyruvate and 10% FBS. To induce differentiation, when cell reached 75% confluency, the medium was switched into DMEM containing 2% horse serum for 7-15 days.

### Transfection and Stable Cell Line Derivation

To construct overexpression vectors, mouse BORG cDNA was amplified by PCR-based methods and subcloned into pCMV-Script A2 vector (Stratagene). All transfections were carried out on ˜90% confluent cells grown in monolayer, using Lipofectamin 2000 (Invitrogen). The overexpression of BORG was verified by RT-PCR. Stable cell lines in C2C12 and C3H10T1/2 cells were derived as described above for N2A cells, but a higher concentration (1 mg/ml) of G418 was used. To super-transfect BORG overexpressing cells with an shRNA against BORG, an shRNA vector containing a puromycin selection marker was used to derive cells stably transfected both with a BORG overexpression vector and an shRNA-expressing vector.

### BMP2 Treatment

Cells were grown in 60 mm dishes in DMEM supplemented with 15% FBS as described above. After reaching the desired confluency, the medium was replaced by DMEM containing 5% FBS in the presence or absence of 300 ng/ml rhBMP2 (GenScript and Sigma). Cells were harvested at indicated time points. The medium (DMEM containing 5% FBS plus or minus 300 ng/ml of rhBMP2) was replaced every 48 hours.

### RNA Preparation

Total RNA was extracted by TRIzol (Molecular Research Center) according to manufacturer's protocol. Briefly, 1 ml of TRIzole Reagent was added per 10 cm² area of the cell culture dish used. The resulting homogenate was mixed with 0.2 ml chloroform per ml of TRIzol and the phases were subsequently separated by centrifugation. The RNA content of the aqueous phase was precipitated by the addition of an equal volume of isopropanol at room temperature, followed by centrifugation at 11,000 g at 4° C. for 15 minutes. The pellets were washed with 75% ethanol and dissolved in RNase-free water and quantified by measuring the optical absorbance at 260 nm wavelength.

### RT-PCR

When possible, RT-PCR primers were designed to flank introns for easy detection of signals coming from any possible genomic DNA contamination of RNA samples. To ensure accurate detection of signals from repeat-element-rich non-coding RNA sequences, RT-PCR reactions were performed using forward primers that were radiolabeled at high specific activity. All primers were optimized and the linear range of amplification for each primer set was determined. The cycle numbers were kept at a minimum to enhance the quantitative accuracy of the reaction. The reactions were performed using the RT-PCR Master Mix (United States Biochemicals), followed by loading on 5% non-denaturing PAGE along with a size marker. RT-PCR reactions on a housekeeping gene (β-actin or GAPDH) were performed in parallel and the results were used for normalization. The gels were exposed to PhosphorImager screens and the bands corresponding to the desired amplicon were quantitated using ImageQuant software. Each experiment was repeated using three biological replicates and at least two technical repeats per replicates. The average value from the different repeats was graphed with two standard deviations as error bars. The averaged value of the control sample in each experiment was set to an arbitrary value of 1, and the value of the rest of the samples was normalized against it.

### Tissue and Primary Cell Expression Pattern Analyses

Male and female mice at E14, E16, E18, and 2, 20, 60 days postnatal were sacrificed according to the guidelines for humane handling of animals. The tissues were harvested and mechanically homogenized and the RNA content was extracted using TRIzol, as described above. The primary cultured neurons, astrocytes and oligodendrocytes were cultured as described. Briefly, primary hippocampal neurons were established in Neurobasal media with a B-27 Supplement (Invitrogen). C57BL/6 E16 mice hippocampi were isolated and dissociated with trypsin as described and plated on poly-L-lysine coated plates or coverslips. Media was replaced every other day and neurons were incubated a minimum of 5 days before use at 5% CO₂ in 37° C. Astrocytes and microglia were cultured from the cerebrum of 1-3 day old C57BL/6 mice similar to Tanabe et al. Cerebrum were isolated and the meninges teased off. Chopped brain tissue was pressed through a Cell Strainer (BD Biosciences) to obtain a single cell suspension that was plated in MEM supplemented with 10% FCS, 2 mM glutamine, 2 mg/ml glucose, 50 U/ml penicillin, and 50 mg/ml streptomycin. At 8 hour post-plating, loosely adherent cells were removed by gentle pipetting. Media was changed and cells split as needed. Primary cultures of brain cells were shaken at a 10 and 20 days for 2 hr at 250 rpm. Shaken media was used to isolate the microglia by pipetting to a single cell suspension and pelleting through a serum cushion for plating. The post-shaking adherent cells after 20 days were >95% astrocytes that were replated for experiments. Cells were harvested and their RNA content was extracted as described above using TRIzol.

### In Situ Hybridization

C2C12 cells were plated in labtek II slide chamber (Nalgene Nunc International). After reaching 70% confluence or in the case of differentiating cells, after 3 days of incubation, the cells were washed in 1× PBS (pH 7.4), and fixed in freshly prepared 4% para-formaldehyde (Electron Microscopy Science) in PBS for 20 min. at room temperature. Fixed cells were rinsed twice for 5 min. in PBS and were permeabilized by incubation in a solution of 1.5% Triton X-100 in PBS for 10 min. at room temperature followed by acetylation in freshly prepared 0.25% acetic acid anhydride in 0.1 M triethanolamine (TEA) for 10 min. at room temperature. Cells were rinsed in 2×SSC for 10 min. at 45° C., air-dried for 5 min. on a slide wanner at 60° C. and were used immediately for hybridization. LNA hybridization probes containing a FITC label at the 5′ end were purchased from Exiqon. The probes were designed to minimize off-target binding and were tested against shRNA-mediated BORG knock down cells in which the level of BORG was not detectable by RT-PCR in order to prove the specificity of the signal. The LNA probes were diluted with hybridization buffer (see below) to a concentration of 100 nM and denatured at 80° C. for 5 min. Hybridization was performed overnight in a humid chamber at 44° C. in 300 μl of hybridization buffer containing 50% (v/v) formamide, 10 mM Tris-HCl pH 7.5, 5 mM EDTA pH 8.0, 2×SSC, 1× Denhardt's solution, 100 μg/ml salmon sperm DNA and 10% dextran sulphate; and 100 nM denatured LNA RNA probes. Non-specific RNA hybridization was eliminated by washing with 2×SSC, 0.1% Tween-20 at 44° C. for 10 min, followed by 3 washes with 0.1×SSC at 65° C. Slides were dehydrated by 5 minutes of sequential incubation in 50%, 75%, 90% and 100% ethanol at RT, and were then air dried and mounted with PROLONG Gold Antifade Reagent (Invitrogen) containing DAPI for nuclear counter-staining. The LNA RNA probe was complementary to nucleotides 2403 to 2424 of BORG RNA (5′-cctttaat attcccatta acct-3′ SEQ ID NO: 3). Images were obtained with a Zeiss Axioskop 2 Plus fluorescence microscope.

Selection of shRNA Constructs

The shRNA constructs were designed as described in Chang et al. Each hairpin sequence was cloned into pGeneclip vectors containing neomycin/G418 resistance gene (Promega) and the accuracy of the sequence of the inserted construct was verified by sequencing. Three shRNA constructs targeting different regions of BORG were created to ensure efficient downregulation of expression and to help distinguish any off-target effects. The chosen sequences did not show significant complementarity to other regions of BORG or elsewhere in the mouse genome as determined by low stringency Blast searches. Three shRNA constructs containing scrambled sequences that did not show complementarity to any region of the mouse genome were designed as controls. The day before transfection, cells were plated at a density of 5×10⁴ cells per well of a 24-well plate. 1 μg of vector DNA was transfected into the cells using lipofectamin 2000. After 48-96 hours of incubation, total cellular RNA from cells was harvested and assayed for reduction in the level of BORG gene expression by RT-PCR to determine the efficiency of knock down by each construct. To obtain stable cell lines, cells were transferred to media containing G418 (1 mg/mL) 48 hours after transfection, followed by 1-2 weeks of incubation until colonies appeared. For each DNA construct, 8 well-isolated colonies were selected using cloning disks. The cells were transferred into 12 well plates containing the G418-containing selection media. The cells were re-selected as described for N2a cells and then were allowed to grow followed by screening for BORG expression by RT-PCR. The sequence of the shRNA constructs is shown below.

shRNA construct covering the region in the vicinity of nucleotide 80 of BORG:

(SEQ ID NO: 4)
GAT CTC GCG TTG ACA GTG AGC GCG CCT CTC TCC TCG
ATA AAG AGT AGT GAA GCC ACA GAT GTA CTC TTT ATC
GAG GAG AGA GGC ATG CCT ACT GCC TCG A.

shRNA construct covering the region in the vicinity of nucleotide 184 of BORG:

(SEQ ID NO: 5)
GAT CTC GCG TTG ACA GTG AGC GAT AGG CCA TGC TCC
AGA TAT TAT AGT GAA GCC ACA GAT GTA TAA TAT CTG
GAG CAT GGC CTA GTG CCT ACT GCC TCG A.

shRNA construct covering the region in the vicinity of nucleotide 941 of BORG:

(SEQ ID NO: 6)
GAT CTC GCG TTG ACA GTG AGC GCT TGG TGA GGA ATG
TAG GTA CTT AGT GAA GCC ACA GAT GTA AGT ACC TAC
ATT CCT CAC CAA ATG CCT ACT GCC TCG A.

### Immunostaining

Cultured cells were fixed in 4% PFA (in 1×PBS) for 20 minutes at RT. After washing three times in PBS at pH 7.2, they were incubated with blocking solution (PBS, 1 g/100 ml BSA and 0.3% triton x-100) at RT for 30 minutes. Primary antibodies were diluted in blocking solution as follows: TuJ1 (mouse; Santa Cruz Biotechnology) 1:500, NeuN (mouse, Santa Cruz Biotechnology) 1:100, Olig2 (rabbit; Santa Cruz Biotechnology) 1:100. For other primary antibodies mentioned below a 1:200 dilution was used: GFAP (C-19, SC-6170, Goat polyclonal IgG, Santa Cruz Biotechnology), GFAP (H-50, SC-9065 rabbit polyclonal IgG, Santa Cruz Biotechnology), GFAP (C-19, SC-6170, Goat polyclonal IgG, Santa Cruz Biotechnology), MAP-2 (H-300, SC-20172, Rabbit polyclonal IgG, Santa Cruz Biotechnology), MAP-2 (5-15, SC-12012, Goat polyclonal IgG, Santa Cruz Biotechnology), Tau (V-20, SC-1996, Goat polyclonal IgG, Santa Cruz Biotechnology). Cultured cells were incubated with the primary antibodies at 4° C. overnight. After several washes with PBS, secondary antibodies were added in blocking solution for 2 hours at RT at 1:1000 dilution. The slides were mounted using ProLong Gold with DAPI mounting Immunostained cultures were examined with Axioskop 2 Plus fluorescent microscope (Carl Zeiss). Controls lacking the primary antibodies were set up in parallel.

### Microarray Analysis

Wild type C2C12 cells and a BORG overexpressing stable C2C12 cell line were cultured in triplicate in 100 mm plates. After reaching 75% confluence, differentiation was induced by switching the cells into the differentiating medium (DMEM supplemented with 2% heatinactivated horse serum). The cells were allowed to differentiate for 5 days. Cells were harvested and total RNA was isolated using TRIzol, as described above. After the purity and integrity of RNA was confirmed using an Agilent 2100 Bioanalyzer, 1 μg of total cellular RNA from each sample was used for target preparation and hybridization as described in the GeneChip Whole Transcript Sense Target Labeling Assay Manual (Affymetrix, Santa Clara, Calif.). Labeled targets were hybridized to the Affymetrix mouse arrays (PN540092). Normalization and probe set summarization was done using AffymetrixPowerTools (APT). Probesets which queried the same gene were averaged to create gene expression values. Differences in gene expression between wild type and BORG overexpressing cultures were determined using Significance Analysis of Microarrays (SAM). Go miner clusters that showed up or down-regulation with a p value below 0.1 were used in generating the final data. To generate the heat maps, genes selected by GO miner and the rest of the genes on the array that showed over two fold change in gene expression were further analyzed manually for relevance to the desired cellular process. Heat maps were generated using Java Treeview or MultiExperiment Viewer.

### BORG Sequence Analysis

The BORG genes in other mammalian species were detected by using a modified Blast algorithm against the sequenced mammalian genomes, and were aligned using LALIGN. The sequence of BORG was blasted against the reference hairpin and mature miRNA databases in miRBase which yielded no matches. A manual folding of BORG in 200 nucleotide-long fragments using RNAStructure similarly did not yield any hairpins that may correspond to an miRNA precursor.

### Cell Growth Analysis Using MTT Assays

Cells were seeded at a density of 10³ cells/ml into 96 well plates and allowed to adhere for 24 h. Each cell type was plated in 8 adjacent wells to ensure repeatability. Cell viability was assessed at desired intervals by adding 20 μl of filter-sterilized MTT (5 mg/ml in PBS) to each well. MTT assays take advantage of the reduction of MTT into formazan by mitochondrial dehydrogenase enzyme, which provides an indirect way of measuring the number of viable cells. Following 3 hours of incubation, media was carefully removed and the purple formazan crystals trapped in cells were dissolved in sterile DMSO (100 μl) by incubation at 37° C. for 10 min. The absorbance at 550 nm was measured using an ELISA microplate reader. The amount of color produced is directly proportional to the number of viable cells. An unused row of 96-well plate wells and a row that contained only the media were used as blanks and for normalizing the absorbance. Each assay was done in 3 independent biological experiments with 8 replicates per sample. The absorbance of the 8 wells containing identical samples was averaged and subtracted from the blank and was normalized to the media-only wells. The growth curve was constructed by plotting the resulting averaged, normalized absorbance against time with two standard deviations as error bars.

While this application has been particularly shown and described with references to preferred embodiments thereof, it will be understood by those skilled in the art that various changes in form and details may be made therein without departing from the scope of the application encompassed by the appended claims. All patents, publications and references cited in the foregoing specification are herein incorporated by reference in their entirety.

1. A method of transdifferentiating a non-neuronal mammalian cell into a neuronal cell comprising:
transfecting the non-neuronal mammalian cell with a nucleic acid that promotes the transdifferentiation of the mammalian cell into a neuronal cell, wherein the nucleic acid is substantially homologous to BORG RNA.

2. The method of claim 1, wherein the non-neuronal mammalian cell is transfected with a vector comprising BORG cDNA, the vector upregulating the expression of BORG RNA in the non-neuronal mammalian cell.

3. The method of claim 2, wherein the vector comprises a promoter operatively linked to the BORG cDNA.

4. The method of claim 1, further comprising the step of culturing the neuronal cell in a growth medium.

5. The method of claim 1, wherein the nucleic acid encodes functioning long non-coding BORG RNA.

6. The method of claim 1, the neuronal cell expressing at least one neural-specific antigen selected from the group consisting of MAP2, TUJ-1, NF200, Tau, synapsin, and nestin.

7. The method of claim 1, the non-neuronal mammalian cell comprising a cell of mesodermic origin.

8. The method of claim 1, the non-neuronal mammalian cell comprising a cell selected from the group consisting of a fibroblast, myoblast, osteoblast, chondroblast, or adipoblast.

9. A transdifferentiated mammalian cell that upregulates expression of BORG RNA, the transdifferentiated mammalian cell expressing at least one neural-specific antigen selected from the group consisting of MAP2, TUJ-1, NF200, Tau, synapsin, and nestin.

10. The transdifferentiated mammalian cell of claim 9 expressing MAP2, TUJ-1, NF200, Tau, synapsin, and nestin.

11. The transdifferentiated mammalian cell of claim 9, the transdifferentiated mammalian cell comprising one or more morphological, physiological or immunological feature(s) of a neuronal cell.

12. The transdifferentiated mammalian cell of claim 9, wherein the transdifferentiated mammalian cell further displays a lack of proliferation.

13. The transdifferentiated mammalian cell of claim 12, the transdifferentiated mammalian cell being transdifferentiated from a cell of mesodermic origin.

14. The transdifferentiated mammalian cell of claim 12, the transdifferentiated mammalian cell being transdifferentiated from a human non-neuronal non-terminally differentiated cell.

15. A therapeutic composition for treating a neurological disorder comprising a therapeutically effective amount of transdifferentiated mammalian cells that upregulate expression of BORG RNA, the transdifferentiated mammalian cells expressing neural-specific antigens selected from the group consisting of MAP2, TUJ-1, NF200, Tau, synapsin, and nestin.

16. The therapeutic composition of claim 15, the transdifferentiated mammalian cells expressing MAP2, TUJ-1, NF200, Tau, synapsin, and nestin.

17. The therapeutic composition of claim 15, the transdifferentiated mammalian cells comprising one or more morphological, physiological or immunological feature(s) of a neuronal cell.

18. The therapeutic composition of claim 15, the transdifferentiated mammalian cells further displaying a lack of proliferation.

19. The therapeutic composition of claim 15, the transdifferentiated mammalian cells being transdifferentiated from cells of mesodermic origin.

20. The therapeutic composition of claim 15, the transdifferentiated mammalian cells being transdifferentiated from human non-neuronal non-terminally differentiated cells.

21-28. (canceled)
